(19) United States
(12) Patent Application Publication
(10) Pub. No.: US 2021/0230364 A1
(43) Pub. Date: Jul. 29, 2021

(54) POLYETHER DERIVATIVES, USES, AND METHODS OF MAKING THE SAME

(71) Applicant: P2 SCIENCE, INC., Woodbridge, CT (US)

(72) Inventors: Ashoke BHATTACHARJEE, Cheshire, CT (US); Patrick FOLEY, New Haven, CT (US); Yonghua YANG, Niantic, CT (US); Anam IKRAM, Brookfield, CT (US); Tania SALAM, New Haven, CT (US); Michael FULLER, Shelton, CT (US)

(21) Appl. No.: 17/133,520

(22) Filed: Dec. 23, 2020

Related U.S. Application Data
(60) Provisional application No. 62/953,850, filed on Dec. 26, 2019.
(60) Provisional application No. 63/043,243, filed on Jun. 24, 2020.
(60) Provisional application No. 63/074,197, filed on Sep. 3, 2020.
(60) Provisional application No. 63/092,406, filed on Oct. 15, 2020.
(60) Provisional application No. 63/125,841, filed on Dec. 15, 2020.

(51) Int. Cl. C08G 65/34; C08K 5/01; C08K 5/092; C08L 65/00

(52) U.S. Cl. CPC C08G 65/34; C08L 65/00; C08K 5/092; C08K 5/01

(57) ABSTRACT

The invention contemplates certain polyethers, polyether derivatives, compositions comprising the same, and methods of making and using those same polymers.

### CROSS-REFERENCE TO RELATED APPLICATIONS

This application is an U.S. non-provisional application which claims priority to, and the benefit of, U.S. Provisional Application No. 62/953,850, filed on Dec. 26, 2019, 63/043,243, filed on Jun. 24, 2020, 63/074,197, filed on Sep. 3, 2020, 63/092,406, filed on Oct. 15, 2020, and 63/125,841, filed on Dec. 15, 2020, the contents of each of which are hereby incorporated by reference in their entireties.

### FIELD

The invention relates to certain polyethers, polyether derivatives (e.g., polymeric alcohols and derivatives thereof), compositions comprising the same, and methods of making and using the same, and methods of analysis thereof. The monomeric precursors of said polymers include, for example, such compounds as citronellol, prenol, isocitronellol and isoprenol.

### BACKGROUND

Liquid polymers have important utility in cosmetic and personal care applications and play critical roles in visual displays, rheology, tribology, and drug delivery. For example, they can be used as lubricants, emollients, or as protective barriers for skin healing and UV protection. Ideally, these materials can be produced in a facile manner, be easily derivatized to modify function, and even more preferably be made from safe and sustainable raw materials.

Liquid polymers commonly used in cosmetic and personal care compositions include polyethylene glycols, mixed glycol polymers, poloxamers (triblock ethylene oxide/propylene oxide copolymers), and silicone polymers. Such liquid polymers are commonly used as emulsifiers, preservatives, stabilizers, fragrance carriers, fragrance retention agents, fragrance fixers, anti-malodor agents, anti-foaming agents, lubricants, emollients, surfactants, protective barriers for skin healing, and as UV protection agents. Hydrocarbons are sometimes combined with such polymers in order to fine-tune the physical and chemical properties of the compositions, such as water content and hydrophobicity.

There is a need for new liquid polymers for use in cosmetic and personal are compositions, which polymers can be produced in a facile manner, be easily mixed with hydrocarbons to modify functions and properties, and preferably be made from safe and sustainable raw materials.

Citronellol, prenol, and isoprenol are all naturally occurring molecules that are also commercially available on a large scale. However, these molecules possess an under-utilized combination of functionalities that allow them to be polymerized and functionalized: an isobutylenic group and an alcohol.

This type of chemistry has been mostly neglected in polymer chemistry. One reason for this could be due to the fact that the polymerization is an equilibrium reaction, and that readily abundant isobutylenic alcohols have not always been available. In recent years, however, the production of citronellol has been increasing rapidly, and one of the largest production routes also uses prenol and isoprenol as intermediates, thereby greatly increasing availability.

WO 2019/028053 discloses novel polymers derived from the naturally occurring and commercially available monomers citronellol, prenol and isoprenol. These monomers were effectively polymerized in a controlled way to yield a number of well-characterized polymeric ether alcohols. In addition, as these polymers as initially formed possess primary alcohol functional groups, WO 2019/028053 further discloses functionalization of the alcohol to derive various ether, ester and other derivative products.

WO 2019/028053 did not disclose means for precisely controlling the average molecular weight (M_{w}), average molecular number (Mₑ) or polydispersity index (PDI) of those polymers, nor did WO 2019/028053 disclose methods for precisely determining these parameters.

Polydispersity is widespread in polymeric systems, since the building blocks are never fully identical but exhibit a continuous spread in size, shape, or surface charge. The variability in microscopic interaction resulting from variations in polydispersity may have a considerable influence on the properties of a polymer, and thus, on its utilities. It is therefore advantageous to precisely control and evaluate the polydispersity of a polymer, and to consider how variations in polydispersity impact the physical and chemical properties of the polymer. By determining the polydispersity-property relationship for a family of related polymers, one may more easily fine tune the desired properties and applications for new polymers.

However, as mentioned above, the equilibrium nature of the polymerization reaction can potentially make it challenging to produce desired ethers on a large scale. There is a need for strategies and methods of production which allow efficient manufacture of these compounds.

### BRIEF SUMMARY

In a surprising advancement in polymer science, the inventors' prior publications US 2017/0283553, U52017/0057940, and WO2019/028053, the contents of each of which are incorporated herein by reference, have taught generally how to prepare polyether polymers and derivatives thereof. These polyethers represent an advance in liquid polymer technology and carry with them many desirable benefits for commercial fields of application.

The present disclosure builds on the inventors' prior work by providing new compounds, compositions, and methods for making and derivatizing such polymers while controlling the molecular weight, molecular number, and/or polydispersity of these polymers.

Polyether, polyamine, and polythioether polymers, and derivatives thereof, have found use as lubricants, emollients, humectants, and surfactants. The polymers and derivatives thereof disclosed herein may be used in cosmetic or specialty chemical formulations and in some instances may be used as naturally derived alternatives to silicone polymers. These polymers can be formulated into various specialty chemical applications, including personal care compositions, in order to alter and improve the function of the product or application performance. The precise functionality of these polymers depends, however, on their size and composition, and hence both on their monomeric distribution, polydispersity, and any further functionalization.

In a first aspect, the present disclosure provides a composition comprising a compound according to Formula I below (Compound 1):

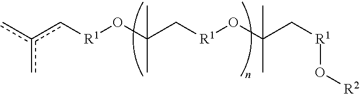

wherein R¹ is optionally substituted C₁-C₁₂ alkyl; R² is H, C₁₋₂₀alkyl, aryl, aryl-C₁₋₂ alkyl, optionally unsaturated alkyl esters or aryl esters, or R² is a moiety:

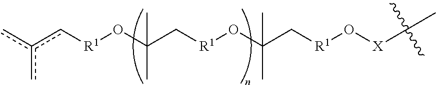

wherein X is a diacyl moiety of formula —C(O)—R³—C(O)—, wherein R³ is optionally

substituted C₁₋₂₂ alkyl, optionally substituted C₂₋₂₂ alkenyl or optionally substituted aryl; and wherein n is an integer between 0 and 10 (e.g., 0 to 4); and wherein the number average or weight average molecular weight of the compounds of Formula I in the composition, optionally exclusive of the group R², is 150 to 2000 Daltons (e.g., 300 to 800 Daltons), and/or wherein the polydispersity (M_{w}/Mₙ) of the compounds of Formula I in the composition (optionally without taking into account the group R²) is in the range of 1 to 5.

It is understood that [symbol] represents an optional double bond (i.e., either a single or double bond), and thus that the terminal group,

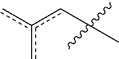

may have any one of the three indicated optional bonds present (i.e., a double bond) or all optional bonds absent (i.e., all single bonds).

In a second aspect, the present disclosure provides a compound of Formula II:

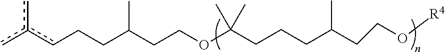

wherein:
R⁴ is selected from Si(C₁₋₄ alkyl)₃, —C(O)—R⁵, —C(O)-A-C(O)—R⁵, -A-C(O)—R⁵, and -A-O—R⁵;
R⁵ is selected from hydrogen, OH, substituted or unsubstituted C₁₋₂₂alkyl, substituted or unsubstituted C₂₋₂₂ alkenyl, substituted or unsubstituted C₂₋₂₂ alkynyl, substituted or unsubstituted C₆₋₁₀ aryl, or wherein R⁵ is a moiety:

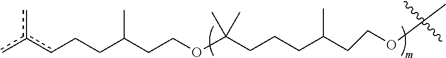

A is selected from substituted or unsubstituted C₁₋₂₀ alkyl, substituted or unsubstituted C₂₋₁₂alkenyl, substituted or unsubstituted C₂₋₁₂ alkynyl, substituted or unsubstituted C₃₋₄₂cycloalkyl, and substituted or unsubstituted C₆₋₁₀ aryl; and

each n and m are independently an integer between 0 and 20;
provided that when R⁴ is —C(O)—R⁵, then R⁵ is not methyl, propyl or oleayl; and
provided that when R⁴ is —C(O)-A-C(O)—R⁵, and R⁵ is a moiety:

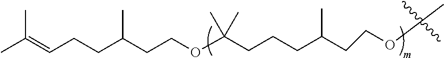

then A is not (CH₂)₄ when n is 0 and m is 1.

In a third aspect, the present disclosure provides a compound of Formula III:

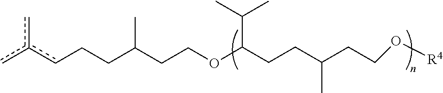

wherein:
R⁴ is selected from hydrogen, Si(C₁₋₄ alkyl)₃, —C(O)—R⁵, —C(O)-A-C(O)—R⁵, -A-C(O)—R⁵, and -A-O—R⁵;
R⁵ is selected from hydrogen, OH, substituted or unsubstituted C₁₋₂₂alkyl, substituted or unsubstituted C₂₋₂₂ alkenyl, substituted or unsubstituted C₂₋₂₂ alkynyl, substituted or unsubstituted C₆₋₁₀ aryl, or wherein R⁵ is a moiety:

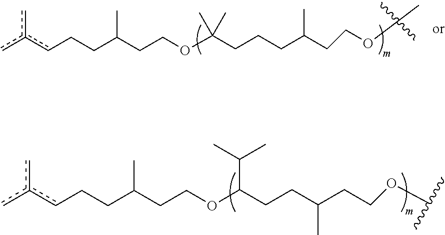

A is selected from substituted or unsubstituted C₁₋₂₀ alkyl, substituted or unsubstituted C₂₋₁₂alkenyl, substituted or unsubstituted C₂₋₁₂ alkynyl, substituted or unsubstituted C₃₋₁₂ cycloalkyl, and substituted or unsubstituted C₆₋₁₀aryl; and each n and m are independently an integer between 0 and 20.

In further aspects, the present disclosure provides further compositions, and methods of manufacturing said compounds and methods of using said compounds. In some embodiments, the compositions further comprise hydrocarbons, for example, linear or branched, saturated or unsaturated hydrocarbons.

### BRIEF DESCRIPTION OF FIGURES

FIG. 1. HPLC chromatogram corresponding to analysis of citronellol polymer acetate composition described in Example 4.

### DETAILED DESCRIPTION

Without wishing to be bound by theory, isobutylenic groups can form ethers with alcohols through an acid catalyzed mechanism. This chemistry has been used in other instances to make ether bonds in organic synthesis.

The equilibrium nature of this reaction can potentially make it challenging to produce these ethers on a large scale. However, the inventors have discovered that with monomer recycling, proper catalyst selection, and highly concentrated reaction conditions, these molecules can reach sufficient degrees of polymerization in order to be used in a number of different applications. Further, these low molecular weight polymers can be further derivatized to reach much higher molecular weights and to achieve new functionality.

In a first aspect, the present disclosure provides a composition comprising a compound (Compound 1) according to Formula I (Composition 1) below:

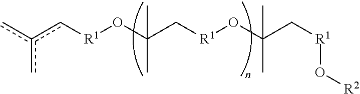

wherein R¹ is optionally substituted C₁₋₁₂ alkyl; R² is H, C₁₋₂₀ alkyl, aryl, aryl-C₁₋₂ alkyl, optionally unsaturated alkyl esters or aryl esters, or R² is a moiety:

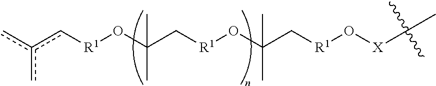

wherein X is a diacyl moiety of formula —C(O)—R³—C(O)—, wherein R³ is optionally

substituted C₁₋₂₂ alkyl, optionally substituted C₂₋₂₂ alkenyl or optionally substituted aryl; and wherein n is an integer between 0 and 10 (e.g., 0 to 4); and wherein the number average or weight average molecular weight of the compounds of Formula I in the composition, exclusive of the group R², is 150 to 2000 Daltons (e.g., 300 to 800 Daltons) and/or wherein the polydispersity (M_{w}/Mₙ) of the compounds of Formula I in the composition (without taking into account the group R²) in the range of 1 to 5.

It is understood that the compositions according to Composition 1 et seq., described herein, comprise a mixture of discrete polymers according to Formula I which vary in the precise value of the integer n. Thus, a composition which comprises only a single polymer according to Formula I having a single value for the integer n is not within the scope of the Composition 1 et seq., and consequently, the Composition 1 et seq. is understood to be a mixture of compounds of Formula I having different values for the integer n, e.g., at least two different compounds of Formula I having different values for the integer n (for example, a mixture which comprises a compound of Formula I wherein n is 0, and a compound of Formula I wherein n is 1.). Typically, in such compositions, substantially all polymers according to Formula I in the composition will have the same groups R¹ and R², that is, the various polymers according to Formula I in the composition will differ only in the value of the integer n. As used in the preceding sentence (and analogously elsewhere herein), the term “substantially all polymers according to Formula I” is understood to recognize that minor synthetic impurities may be present in which R¹ and/or R² differ from that of the bulk of the composition (e.g., owing to minor impurities in starting materials, minor side-products in the synthesis, or minor amounts of unreacted intermediates, which may be present despite efforts at purification).

In further embodiments of the first aspect, the disclosure provides any of the following:
1.1 Composition 1, wherein R¹ is optionally substituted linear C₁-C₁₂ alkyl or optionally substituted branched C₁-C₁₂ alkyl.
1.2 Composition 1, wherein R¹ is unsubstituted linear C₁-C₁₂ alkyl or unsubstituted branched C₃-C₁₂ alkyl.
1.3 Composition 1, wherein R¹ is unsubstituted linear C₁-C₁₂ alkyl.
1.4 Composition 1, wherein R¹ is unsubstituted branched C₃-C₁₂ alkyl.
1.5 Composition 1, wherein R¹ is CH₂.
1.6 Composition 1, wherein R¹ is unsubstituted branched or linear C₆ alkyl.
1.7 Composition 1, wherein R¹ is CH₂CH₂CH(CH₃)CH₂CH₂.
1.8 Composition 1 or any of 1.1-1.7, wherein R² is H.
1.9 Composition 1 or any of 1.1-1.7, wherein R² is alkyl (e.g., lower alkyl (e.g., C₁₋₆), or C₁₋₁₂).
1.10 Composition 1 or any of 1.1-1.7, wherein R² is selected from methyl, ethyl, propyl, isopropyl, n-butyl, isobutyl, sec-butyl, tert-butyl, n-pentyl, n-hexyl, n-octyl, or n-decyl.
1.11 Composition 1 or any of 1.1-1.7, wherein R² is arylC₁₋₂ alkyl (e.g., benzyl or phenethyl).
1.12 Composition 1 or any of 1.1-1.7, wherein R² is aryl (e.g., phenyl).
1.13 Composition 1 or any of 1.1-1.7, wherein R² is an optionally unsaturated alkyl ester (e.g., C(O)—C₁₋₂₀ alkyl, or C(O)—C₁₋₆ alkyl).
1.14 Composition 1 or any of 1.1-1.7, wherein R² is C(O)—C₁₋₆ alkyl, optionally wherein R² is C(O)—C₁₋₅ alkyl, C(O)—C₁₋₄ alkyl, C(O)—C₁₋₃ alkyl or C(O)—C₁₋₂ alkyl.
1.15 Composition 1 or any of 1.1-1.7, wherein R² is C(O)—C₁₋₆ alkyl and said C₁₋₆ alkyl is selected from methyl, ethyl, propyl, isopropyl, n-butyl, isobutyl, sec-butyl, tert-butyl, n-pentyl, or n-hexyl.
1.16 Composition 1 or any of 1.1-1.7, wherein R² is optionally unsaturated C(O)—C₇₋₂₀ alkyl, optionally wherein R² is optionally unsaturated C(O)—C₁₀₋₂₀ alkyl, C(O)—C₁₂₋₂₀ alkyl, C(O)—C₁₄₋₂₀ alkyl or C(O)—C₁₆₋₁₈ alkyl, or C(O)—C₁₇ alkyl.
1.17 Composition 1 or any of 1.1-1.7, wherein R² is mono-unsaturated C(O)—C₇₋₂₀ alkyl, optionally wherein R² is mono-unsaturated C(O)—C₁₀₋₂₀ alkyl, C(O)—C₁₂₋₂₀ alkyl, C(O)—C₁₄₋₂₀ alkyl or C(O)—C₁₆₋₁₈ alkyl, or C(O)—C₁₇ alkyl (e.g., oleayl).
1.18 Composition 1 or any of 1.1-1.7, wherein R² is saturated C(O)—C₇₋₂₀ alkyl, optionally wherein R² is saturated C(O)—C₁₀₋₂₀ alkyl, C(O)—C₁₂₋₂₀ alkyl, C(O)—C_{14_20} alkyl or C(O)—C₁₆₋₁₈ alkyl, or C(O)—C₁₇ alkyl.
1.19 Composition 1 or any of 1.1-1.18, wherein the R² substituent is a fatty acyl chain.
1.20 Composition 1 or any of 1.1-1.7, wherein R² is an aryl ester (e.g., C(O)-aryl), for example, benzoyl.
1.21 Composition 1 or any of 1.1-1.18, wherein the R² substituent further comprises a cationic or anionic moiety (e.g., wherein R² is alkyl, aryl, alkyl ester, or aryl ester wherein said alkyl or aryl is substituted with a cationic (e.g., quaternary ammonium) or anionic (e.g., carboxylic acid or sulfonic acid) moiety.
1.22 Composition 1 or any of 1.1-1.18, wherein R² is a polyether moiety, e.g., wherein the R² substituent comprises a polyethylene glycol chain.
1.23 Composition 1 or any of 1.1 et seq., wherein n is 0 to 8, or 1 to 7.
1.24 Composition 1 or any of 1.1 et seq., wherein n is 0 to 6, or 1 to 5.
1.25 Composition 1 or any of 1.1 et seq., wherein n is 0 to 4, or 1 to 4.
1.26 Composition 1 or any of 1.1 et seq., wherein n is 0, 1, 2, 3, or 4.
1.27 Composition 1 or any of 1.1 et seq., wherein n is 0, 1 or 2.
1.28 Composition 1 or any of 1.1 et seq., wherein R¹ is CH₂CH₂CH(CH₃)CH₂CH₂, n is 1-3 (e.g., 1-2) and R² is an optionally unsaturated alkyl ester (e.g., C(O)—C₁₋₂₀ alkyl, or C(O)—C₁₋₆ alkyl).
1.29 Composition 1.28, wherein R² is C(O)—C₁₋₆ alkyl and said C₁₋₆ alkyl is selected from methyl, ethyl, propyl, isopropyl, n-butyl, isobutyl, sec-butyl, tert-butyl, n-pentyl, or n-hexyl.
1.30 Composition 1.28, wherein R² is mono-unsaturated C(O)—C₇₋₂₀ alkyl, optionally wherein R² is mono-unsaturated C(O)—C_{10_20} alkyl, C(O)—C_{12_20} alkyl, C(O)—C_{14_20} alkyl or C(O)—C₁₆₋₁₈ alkyl, or C(O)—C₁₇ alkyl (e.g., oleayl).
1.31 Composition 1, or any of 1.1 et seq., wherein R² is an anti-aging moiety, UV-absorbing moiety, anti-oxidant moiety, hydrophobic (lipophilic) moiety, or hydrophilic moiety, as described herein.
1.32 Composition 1 or any of 1.1 et seq., wherein R² is not H, e.g., R² is alkyl (e.g., lower alkyl (e.g., C₁₋₆ alkyl), or C₁₋₁₂ alkyl), aryl (e.g., phenyl), C₁-C₂ alkylaryl (e.g., benzyl), optionally unsaturated alkyl esters (e.g., C(O)—C₁₋₂₀ alkyl), or aryl esters (e.g., C(O)-aryl), and further embodiments of R² as described hereinabove.

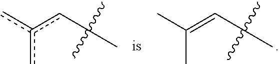

1.33 Composition 1 or any of 1.1 et seq., wherein the terminal group

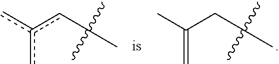

1.34 Composition 1 or any of 1.1 et seq., wherein the terminal group

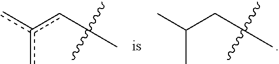

1.35 Composition 1 or any of 1.1 et seq., wherein the terminal group
1.36 Composition 1 or any of 1.1 et seq., wherein the Compound of Formula I is:

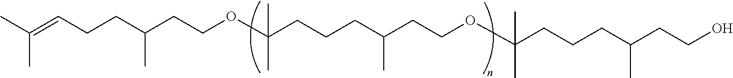

where n: 0-10 (e.g., 0, 1 or 2).
1.37 Composition 1 or any of 1.1 et seq., wherein the Compound of Formula I is:

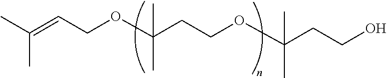

where n: 0-10 (e.g., 0, 1 or 2).
1.38 Composition 1 or any of 1.1 et seq., wherein the Compound of Formula I is:

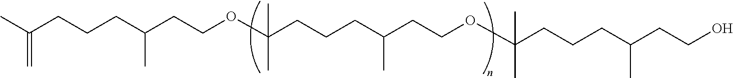

where n: 0-10 (e.g., 0, 1 or 2).
1.39 Composition 1 or any of 1.1 et seq., wherein the Compound of Formula I is:

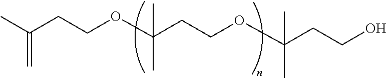

where n: 0-10 (e.g., 0, 1 or 2).
1.40 Composition 1 or any of 1.1 et seq., wherein the Compound of Formula I is:

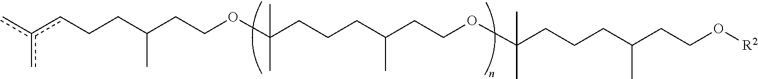

where n: 0-10 (e.g., 1-7, 1-4, 1-3 or 0, 1, 2, or 3), and wherein R² is as defined in any preceding embodiment, optionally wherein the Compound of Formula I is:

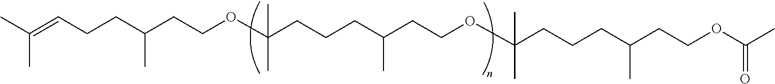

wherein n is 0-10, 1-7, 1-4, 1-3, or 1, 2 or 3.
1.41 Composition 1 or any of 1.1 et seq., wherein the Compound of Formula I is:

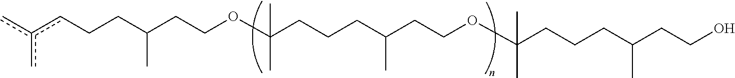

where n: 0-10 (e.g., 1-7, 1-4, 1-3 or 1, 2 or 3), optionally wherein the Compound of Formula I is:

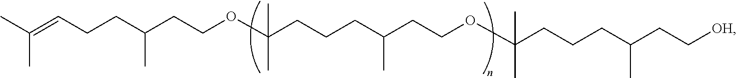

wherein n is 0-10, 1-7, 1-4, 1-3, or 1, 2 or 3.
1.42 Composition 1 or any of 1.1 et seq., wherein the Compound of Formula I is:

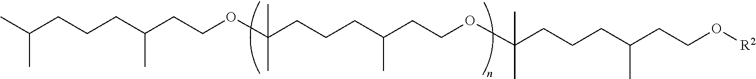

where n: 0-10 (e.g., 1, 2 or 3), and wherein R² is as defined in any preceding embodiment, optionally wherein the Compound of Formula I is:

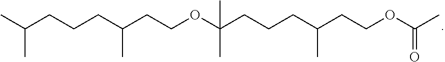 [SMILES: CC(=O)OCCC(C)CCCC(C)(C)OCCC(C)CCCC(C)C]

1.43 Composition 1 or any of 1.1 et seq., wherein the Compound of Formula I is:

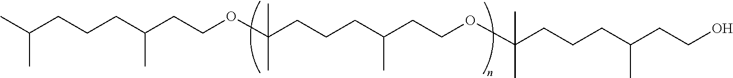

where n: 0-10 (e.g., 1, 2 or 3), optionally wherein the Compound of Formula I is:

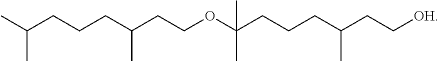 [SMILES: CC(C)CCCC(C)CCOC(C)(C)CCCC(C)CCO]

1.44 Composition 1 or any of 1.1 et seq., wherein the Compound of Formula I is enantiomerically enriched, e.g., having a diastereomeric excess (e.g., an enantiomeric excess (e.e.)) of greater than 70%.
1.45 Composition 1.44, wherein the compound of Formula I has a diastereomeric excess (e.g., an enantiomeric excess (e.e.)) of greater than 80%, or greater than 85%, or greater than 90%, or greater than 95%, or greater than 97% or greater than 99%.
1.46 Composition 1.44 or 1.45, wherein each chiral carbon of the compound of Formula I, exclusive of the group R², has the (R)-configuration, optionally wherein each chiral carbon of the group R² also has the (R)-configuration.
1.47 Composition 1.44 or 1.45, wherein each chiral carbon of the compound of Formula I, exclusive of the group R², has the (S)-configuration, optionally wherein each chiral carbon of the group R² also has the (S)-configuration.
1.48 Composition 1.44 or 1.45, wherein each chiral carbon of the compound of Formula I, exclusive of the group R², has the (R)-configuration, and wherein each chiral carbon of the group R² has the (S)-configuration.
1.49 Composition 1.44 or 1.45, wherein each chiral carbon of the compound of Formula I, exclusive of the group R², has the (S)-configuration, and wherein each chiral carbon of the group R² has the (R)-configuration.
1.50 Composition 1 or any of 1.1 et seq., wherein the composition comprises a single compound of Formula I according to any one of the compounds described in Composition 1 or 1.1-1.49, the compound being present to an extent of greater than 40%, e.g., greater than 50%, greater than 60%, or greater than 70%, or greater than 80%, or greater than 90%, or 40-70% or 40-60%, or 40-50%, said percent being measured either as the number percent of the molecules in the composition or as the weight percent of the total weight of the composition.
1.51 Composition 1 or any of 1.1 et seq., wherein the composition comprises one or more compounds of Formula I (e.g., from one up to ten specific compounds), wherein each compound is independently a compound described in Composition 1 or 1.1-1.49, each compound being present in an amount of at least 1% and up to 90%, e.g., 5 to 50%, or 5 to 40% or 5 to 30%, or 5 to 25%, or 5 to 20% or 5 to 15%, or 5 to 10%, or 40 to 50%, or 30 to 40% or 20 to 30%, or 10 to 20%, or 1 to 10%, or 1 to 5%, or 1 to 2%, said percent being measured either as the number percent of the molecules in the composition or as the weight percent of the total weight of the composition.
1.52 Composition 1 or any of 1.1 et seq., wherein the number average molecular weight (Mₙ) of the compounds of Formula I in the composition, optionally exclusive of the group R², is 150 to 2000 Daltons.
1.53 Composition 1.52, wherein the number average molecular weight (Mₙ) of the compounds of Formula I in the composition, optionally exclusive of the group R², is 300 to 1900 Daltons, e.g., 300 to 1600 Daltons, or 300 to 1300 Daltons, or 300 to 1100 Daltons, or 300 to 1000 Daltons, or 300 to 800 Daltons, or 300 to 700 Daltons, or 300 to 500 Daltons, or 400 to 1000 Daltons, or 400 to 700 Daltons, or 600 to 1100 Daltons, or 600 to 1000 Daltons, or 600 to 800 Daltons or about 500 Daltons, or about 414 Daltons.
1.54 Composition 1 or any of 1.1 et seq., wherein the weight average molecular weight (M_{w}) of the compounds of Formula I in the composition, optionally exclusive of the group R², is 150 to 2000 Daltons.
1.55 Composition 1.54, wherein the weight average molecular weight (M_{w}) of the compounds of Formula I in the composition, optionally exclusive of the group R², is 300 to 1900 Daltons, e.g., 300 to 1600 Daltons, or 300 to 1300 Daltons, or 300 to 1100 Daltons, or 300 to 1000 Daltons, or 300 to 800 Daltons, or 300 to 700 Daltons, or 300 to 500 Daltons, or 400 to 1000 Daltons, or 400 to 700 Daltons, or 600 to 1100 Daltons, or 600 to 1000 Daltons, or 600 to 800 Daltons, or about 438 Daltons.
1.56 Composition 1 or any of 1.1 et seq., wherein the polydispersity (M_{w}/Mₙ) of the compounds of Formula I in the composition (optionally without taking into account the mass of the group R²) is in the range of 1 to 5.
1.57 Composition 1.56, wherein the polydispersity (M_{w}/Mₙ) is in the range of 1 to 4, or 1 to 3, or 1 to 2.5, or 1 to 2, or 1 to 1.5, or about 1 to 1.25, or about 1, or 1.5 to 3.5, or 1.5 to 2.5, or about 1.5, or 2 to 4, or 2 to 3, or 2 to 2.5, or about 2, or 1 to 1.25, or 1 to 1.20, or 1 to 1.15, or 1 to 1.10, or about 1.06.
1.58 Composition 1 or any of 1.1 et seq., wherein the compounds of Formula I in the composition have an average n value of 0 to 8, measured either as a weight average or number average.
1.59 Composition 1 or any of 1.1 et seq., wherein the compounds of Formula I in the composition have an average n value of 0 to 6, measured either as a weight average or number average.
1.60 Composition 1 or any of 1.1 et seq., wherein the compounds of Formula I in the composition have an average n value of 0 to 5, measured either as a weight average or number average.
1.61 Composition 1 or of 1.1 et seq., wherein the compounds of Formula I in the composition have an average n value of about 0, about 1, about 2, about 3, or about 4, measured either as a weight average or number average.
1.62 Composition 1 or any of 1.1 et seq., wherein the compounds of Formula I in the composition have an average n value of about 0, about 1 or about 2, measured either as a weight average or number average.
1.63 Composition 1.62, wherein the compounds of Formula I in the composition have an average n value of about 0.5, e.g., about 0.52, measured as the weight average.
1.64 Composition 1 or any of 1.1 et seq., wherein the compounds of Formula I in the compositions are all compounds of the structure:

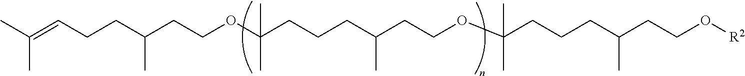

wherein R² is acetyl and n is from 0 to 8 (e.g., 0 to 4 or 1 to 7).
1.65 Composition 1.64, wherein at least 95% of the compounds of Formula I by weight have an n value of 0, 1, 2 3 or 4, e.g., at least 97% or at least 98% or at least 99% of said compounds.
1.66 Composition 1.65, wherein at least 90% of the compounds of Formula I by weight have an n value of 0, 1 or 2, e.g., 90-98% or 95-98% of said compounds.
1.67 Composition 1.66, wherein at least 70% of the compounds of Formula I by weight have an n value of 0 or 1, e.g., at least 80%, or 80-90% or 80-85% of said compounds.
1.68 Composition 1.67, wherein at least 30% of the compounds of Formula I by weight have an n value of 0, e.g., at least 40%, or 40-60% or 45-55% of said compounds.
1.69 Composition I, or any of 1.1-1.68, wherein the composition further comprises one or more hydrocarbons.
1.70 Composition 1.69, wherein the hydrocarbons comprise linear or branched aliphatic hydrocarbons having from 7 to 30 carbon atoms, e.g., 7 to 25 carbon atoms, or 7 to 23 carbon atoms, or 7 to 12 carbon atoms, or 12 to 15 carbon atoms, or 15 to 18 carbon atoms, or 18 to 21 carbon atoms, or 21 to 23 carbon atoms, or 23 to 28 carbon atoms, or any combination thereof.
1.71 Composition 1.69, wherein the hydrocarbons comprise branched or unbranched cyclic aliphatic hydrocarbons having from 5 to 20 carbon atoms, e.g., 5 to 16 carbon atoms, or 5 to 12 carbon atoms, or 5 to 10 carbon atoms, or 5 to 8 carbon atoms, or 8 to 10 carbon atoms, or 10 to 12 carbon atoms, or 12 to 16 carbon atoms, or 16 to 20 carbon atoms, or any combination thereof.
1.72 Any one of Compositions 1.69-1.71, wherein the hydrocarbons comprise saturated hydrocarbons, monounsaturated hydrocarbons, polyunsaturated hydrocarbons, or some combination thereof.
1.73 Any one of Compositions 1.69-1.72, wherein the hydrocarbons do not comprise aromatic hydrocarbons, and/or do not comprise cyclic hydrocarbons, e.g., wherein the composition does not comprise aromatic hydrocarbons and/or cyclic hydrocarbons.
1.74 Any one of Compositions 1.69-1.73, wherein the one or more hydrocarbons are selected from heptane, octane, nonane, decane, undecane, dodecane, isododecane, tridecane, tetradecane, pentadecane, hexadecane, heptadecane, octadecane, nonadecane, henicosane, docosane, and tricosane, and any saturated linear or saturated branched isomer thereof.
1.75 Composition 1.74, wherein the one or more hydrocarbons are selected from undecane, tridecane, or a mixture thereof.
1.76 Any of Compositions 1.69-1.75, wherein the one or more hydrocarbons comprises a net amount of 0.1 to 95% by weight of the composition, e.g., 1 to 90%, or 5 to 80%, or 10 to 70%, or 15 to 60%, or 5 to 60%, or 10 to 50%, or 15 to 40%, or 1 to 30%, or 1 to 25%, or 5 to 20%, or 20 to 40%, or 40 to 60%, or 60 to 80%, or 80-95%, or 25 to 75%, or about 50%, by weight of the composition.
1.77 Any of Compositions 1.69-1.75, wherein the composition consists of, or consists essentially of, from 5 to 95% by weight of the polymer mixture of compounds of Formula I described in any one of embodiments 1.1-1.68, and the balance (from 95 to 5%) by weight of the hydrocarbon mixture described in any of embodiments 1.68-1.76.

It is understood that the compounds of Formula I consist of a polymeric backbone that may be formed via a controlled homopolymerization reaction between monomeric units to form a compound of Formula I wherein R² is H. In a subsequent reaction (or more than one), a derivative may be formed wherein R² is a moiety other than H. The resulting polymer derivative will necessarily substantially retain the polymeric structural features—for example the values of n and the identities and structural relationships among and between the various monomeric units of the polymer backbone—from the previously formed hydroxylic polymer. As a result, when applied to compounds of Formula I wherein R² is a moiety other than H, it is understood that figures for average molecular weight and polydispersity may be described based on the polymer backbone of the molecule excluding the group R². For example, measurements and calculations of molecular weight and polydispersity can be made on the alcoholic polymer (R² is H) prior to further derivatization, and the resulting values can be extrapolated to later-derived derivatives (wherein R² is a moiety other than H).

In a second aspect, the present disclosure provides a compound of Formula II (Compound 2):

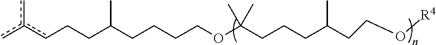

wherein:
R⁴ is selected from Si(C₁₋₄ alkyl)₃, —C(O)—R⁵, —C(O)-A-C(O)—R⁵, -A-C(O)—R⁵, and -A-O—R⁵;
R⁵ is selected from hydrogen, OH, substituted or unsubstituted C₁₋₂₂alkyl, substituted or unsubstituted C₂₋₂₂ alkenyl, substituted or unsubstituted C₂₋₂₂ alkynyl, substituted or unsubstituted C₆₋₁₀ aryl, or wherein R⁵ is a moiety:

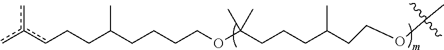

A is selected from substituted or unsubstituted C₁₋₂₀ alkyl, substituted or unsubstituted C₂₋₁₂ alkenyl, substituted or unsubstituted C₂₋₁₂alkynyl, substituted or unsubstituted C₃₋₁₂cycloalkyl, and substituted or unsubstituted C₆₋₁₀ aryl; and each n and m are independently an integer between 0 and 20;

provided that when R⁴ is —C(O)—R⁵, then R⁵ is not methyl, propyl or oleayl; and

provided that when R⁴ is —C(O)-A-C(O)—R⁵, and R⁵ is a moiety:

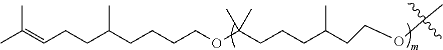

then A is not (CH₂)₄ when n is 0 and m is 1.

In further embodiments of the second aspect, the disclosure provides any of the following:
2.1 Compound 2, wherein R⁴ is Si(C₁₋₄ alkyl)₃.
2.2 Compound 2.1, wherein each C₁₋₄ alkyl is independently selected from methyl, ethyl, propyl, isopropyl, n-butyl, isobutyl, sec-butyl, and tert-butyl.
2.3 Compound 2.2, wherein each C₁₋₄ alkyl is independently selected from methyl and ethyl.
2.4 Compound 2, wherein R⁴ is —C(O)—R⁵.
2.5 Compound 2, wherein R⁴ is —C(O)-A-C(O)—R⁵.
2.6 Compound 2, wherein R⁴ is -A-C(O)—R⁵.
2.7 Compound 2, wherein R⁴ is -A-O—R⁵.
2.8 Compound 2, or any of 2.4-2.7, wherein R⁵ is a moiety:

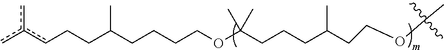

2.9 Compound 2.8, wherein m is an integer between 0 and 10, e.g., between 0 and 8, or between 0 and 6, or between 0 and 4.
2.10 Compound 2.9, wherein m is selected from 0, 1, 2 and 3.
2.11 Compound 2, or any of 2.4-2.7, wherein R⁵ is substituted or unsubstituted C₁₋₂₂alkyl, e.g., substituted or unsubstituted C₁₋₁₆alkyl, or substituted or unsubstituted C₁₋₁₂alkyl, or substituted or unsubstituted C₁₋₆ alkyl.
2.12 Compound 2.11 R⁵ is selected from unsubstituted methyl, ethyl, propyl, isopropyl, n-butyl, isobutyl, sec-butyl, tert-butyl, n-pentyl, n-hexyl, n-octyl, or n-decyl.
2.13 Compound 2, or any of 2.4-2.7, wherein R⁵ is substituted or unsubstituted C₂₋₂₂alkenyl, e.g., substituted or unsubstituted C₂₋₁₆ alkenyl, or substituted or unsubstituted C₂-12alkenyl, or substituted or unsubstituted C₂₋₆ alkenyl.
2.14 Compound 2.13, wherein R⁵ is selected from mono-unsaturated and unsubstituted C₇₋₂₀ alkenyl, C₁₀₋₂₀ alkenyl, C₁₂₋₂₀ alkenyl, C₁₄₋₂₀ alkenyl and C₁₆₋₁₈ alkenyl.
2.15 Compound 2.13, wherein R⁵ is selected from di-unsaturated and unsubstituted C₇₋₂₀ alkenyl, C₁₀₋₂₀ alkenyl, C₁₂₋₂₀ alkenyl, C₁₄₋₂₀ alkenyl and C₁₆₋₁₈ alkenyl.
2.16 Compound 2, or any of 2.4-2.7, wherein R⁵ is substituted or unsubstituted C₂₋₂₂alkynyl, e.g., substituted or unsubstituted C₂₋₁₆alkynyl, or substituted or unsubstituted C₂₋₁₂alkynyl, or substituted or unsubstituted C₂₋₆ alkynyl.
2.17 Compound 2, or any of 2.4-2.7, wherein R⁵ is substituted or unsubstituted C₆₋₁₀aryl, e.g., substituted or unsubstituted phenyl or napthyl.
2.18 Compound 2, or any of 2.4-2.7, wherein R⁵ is OH.
2.19 Compound 2 or any of 2.5-2.18, wherein A is substituted or unsubstituted C₁₋₂₀alkyl, e.g., substituted or unsubstituted C₁₋₁₆alkyl, or substituted or unsubstituted C₁₋₁₂alkyl, or substituted or unsubstituted C₁₋₆ alkyl.
2.20 Compound 2.19, wherein A is (CH₂)_{y} wherein y is an integer from 1 to 6, e.g., wherein y is 2, 3, 4, 5 or 6.
2.21 Compound 2.20, wherein A is (CH₂)₃ or (CH₂)₆.
2.22 Compound 2.20, wherein A is (CH₂)₂.
2.23 Compound 2 or any of 2.1-2.22, wherein n is 0 to 8.
2.24 Compound 2 or any of 2.1-2.22, wherein n 0 to 6.
2.25 Compound 2 or any of 2.1-2.22, wherein n is 0 to 4.
2.26 Compound 2 or any of 2.1-2.22, wherein n is 0, 1, 2, 3, or 4.
2.27 Compound 2 or any of 2.1-2.22, wherein n is 0, 1 or 2.

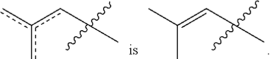

2.28 Compound 2 or any of 2.1-2.27, wherein the terminal group

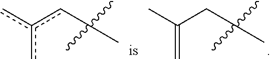

2.29 Compound 2 or any of 2.1-2.27, wherein the terminal group

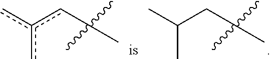

2.30 Compound 2 or any of 2.1-2.27, wherein the terminal group
2.31 Compound 2 or any of 2.1-2.30, wherein the compound of Formula II is enantiomerically enriched, e.g., having a diastereomeric excess (e.g., an enantiomeric excess (e.e.)) of greater than 70%.
2.32 Compound 2.31, wherein the compound of Formula II has a diastereomeric excess (e.g., an enantiomeric excess (e.e.)) of greater than 80%, or greater than 85%, or greater than 90%, or greater than 95%, or greater than 97% or greater than 99%.
2.33 Compound 2.31 or 2.32, wherein each chiral carbon of the compound of Formula II, exclusive of the group R⁴, has the (R)-configuration, optionally wherein each chiral carbon of the group R⁴ also has the (R)-configuration.
2.34 Compound 2.31 or 2.32, wherein each chiral carbon of the compound of Formula II, exclusive of the group R⁴, has the (S)-configuration, optionally wherein each chiral carbon of the group R⁴ also has the (S)-configuration.
2.35 Compound 2.31 or 2.32, wherein each chiral carbon of the compound of Formula II, exclusive of the group R⁴, has the (R)-configuration, and wherein each chiral carbon of the group R⁴ has the (S)-configuration.
2.36 Compound 2.31 or 2.32, wherein each chiral carbon of the compound of Formula II, exclusive of the group R⁴, has the (S)-configuration, and wherein each chiral carbon of the group R⁴ has the (R)-configuration.
2.37 Compound 2, or any of 2.1-2.36, wherein the compound of Formula II is selected from the group consisting of:

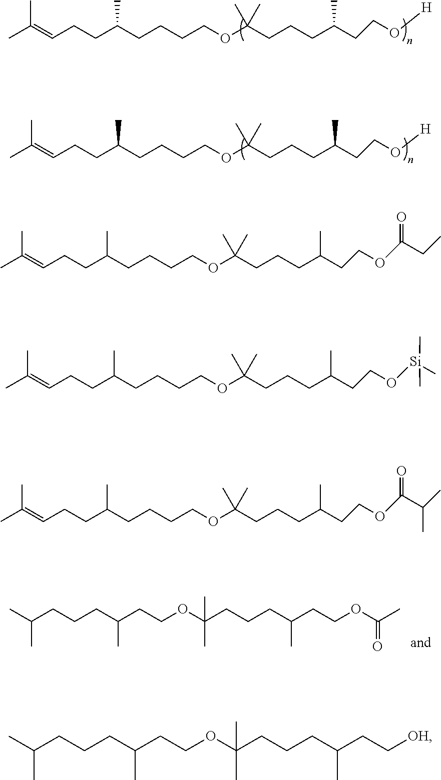

wherein n is an integer from 0 to 10, e.g., 0 to 6, or 0 to 4, or 0, 1, 2 or 3.
2.38 A Composition comprising any one of Compound 2 or any of 2.1-2.37.
2.39 The Composition 2.38, wherein the composition comprises a single compound of Formula II according to Compound 2 or any of 2.1-2.37, wherein the compound is present to an extent of greater than 40%, e.g., greater than 50%, greater than 60%, or greater than 70%, or greater than 80%, or greater than 90%, or 40-70% or 40-60%, or 40-50%, said percent being measured either as the number percent of the molecules in the composition or as the weight percent of the total weight of the composition.
2.40 Composition 2.39, wherein the composition comprises one or more compounds of Formula II (e.g., from one up to ten specific compounds), wherein each compound is independently a compound according to Compound 2 or 2.1-2.37, each compound being present in an amount of at least 1% and up to 90%, e.g., 5 to 50%, or 5 to 40% or 5 to 30%, or 5 to 25%, or 5 to 20% or 5 to 15%, or 5 to 10%, or 40 to 50%, or 30 to 40% or 20 to 30%, or 10 to 20%, or 1 to 10%, or 1 to 5%, or 1 to 2%, said percent being measured either as the number percent of the molecules in the composition or as the weight percent of the total weight of the composition.
2.41 Any one of Compositions 2.39 to 2.40, wherein the number average molecular weight (Mₙ) of the compounds of Formula II in the composition, optionally exclusive of the group R⁴, is 150 to 2000 Daltons.
2.42 Composition 2.41, wherein the number average molecular weight (Mₙ) of the compounds of Formula II in the composition, optionally exclusive of the group R⁴, is 300 to 1900 Daltons, e.g., 300 to 1600 Daltons, or 300 to 1300 Daltons, or 300 to 1100 Daltons, or 300 to 1000 Daltons, or 300 to 800 Daltons, or 300 to 700 Daltons, or 300 to 500 Daltons, or 400 to 1000 Daltons, or 400 to 700 Daltons, or 600 to 1100 Daltons, or 600 to 1000 Daltons, or 600 to 800 Daltons or about 500 Daltons, or about 414 Daltons.
2.43 Any one of Compositions 2.39 to 2.42, wherein the weight average molecular weight (M_{w}) of the compounds of Formula II in the composition, optionally exclusive of the group R⁴, is 150 to 2000 Daltons.
2.44 Composition 2.43, wherein the weight average molecular weight (M_{w}) of the compounds of Formula II in the composition, optionally exclusive of the group R⁴, is 300 to 1900 Daltons, e.g., 300 to 1600 Daltons, or 300 to 1300 Daltons, or 300 to 1100 Daltons, or 300 to 1000 Daltons, or 300 to 800 Daltons, or 300 to 700 Daltons, or 300 to 500 Daltons, or 400 to 1000 Daltons, or 400 to 700 Daltons, or 600 to 1100 Daltons, or 600 to 1000 Daltons, or 600 to 800 Daltons, or about 438 Daltons.
2.45 Any one of Compositions 2.39 to 2.45, wherein the polydispersity (M_{w}/Mₙ) of the compounds of Formula II in the composition (optionally without taking into account the mass of the group R⁴) is in the range of 1 to 5.
2.46 Composition 2.45, wherein the polydispersity (M_{w}/Mₙ) is in the range of 1 to 4, or 1 to 3, or 1 to 2.5, or 1 to 2, or 1 to 1.5, or about 1 to 1.25, or about 1, or 1.5 to 3.5, or 1.5 to 2.5, or about 1.5, or 2 to 4, or 2 to 3, or 2 to 2.5, or about 2, or 1 to 1.25, or 1 to 1.20, or 1 to 1.15, or 1 to 1.10, or about 1.06.
2.47 Any one of Compositions 2.39 to 2.46, wherein the compounds of Formula II in the composition have an average n value of 0 to 8, measured either as a weight average or number average.
2.48 Composition 2.47, wherein the compounds of Formula II in the composition have an average n value of 0 to 6, measured either as a weight average or number average.
2.49 Composition 2.47, wherein the compounds of Formula II in the composition have an average n value of 0 to 5, measured either as a weight average or number average.
2.50 Composition 2.47, wherein the compounds of Formula II in the composition have an average n value of about 0, about 1, about 2, about 3, or about 4, measured either as a weight average or number average.
2.51 Composition 2.47, wherein the compounds of Formula II in the composition have an average n value of about 0, about 1 or about 2, measured either as a weight average or number average.

It is understood that the compounds of Formula II consist of a citronellol polymer backbone that may be formed via a controlled homopolymerization reaction of citronellol to form the compound of Formula II wherein R⁴ is H. In a subsequent reaction (or more than one), a derivative may be formed wherein R⁴ is a moiety other than H. The resulting citronellol homopolymer derivative will necessarily substantially retain the polymeric structural features—for example the values of n and the structural relationships among and between the citronellol monomer units of the polymer backbone—from the previously formed hydroxylic polymer. As a result, when applied to compounds of Formula II wherein R⁴ is a moiety other than H, it is understood that figures for average molecular weight and polydispersity may be described based on the polymer backbone of the molecule excluding the group R⁴. For example, measurements and calculations of molecular weight and polydispersity can be made on the alcoholic polymer (R⁴ is H) prior to further derivatization, and the resulting values can be extrapolated to later-derived derivatives (wherein R⁴ is a moiety other than H).

In a third aspect, the present disclosure provides a compound of Formula III (Compound 3):

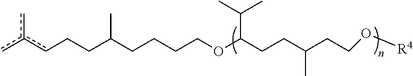

wherein:
R⁴ is selected from hydrogen, Si(C₁₋₄alkyl)₃, —C(O)—R⁵, —C(O)-A-C(O)—R⁵, -A-C(O)—R⁵, and -A-O—R⁵;
R⁵ is selected from hydrogen, OH, substituted or unsubstituted C₁₋₂₂ alkyl, substituted or unsubstituted C₂₋₂₂alkenyl, substituted or unsubstituted C₂₋₂₂alkynyl, substituted or unsubstituted C₆₋₁₀aryl, or wherein R⁵ is a moiety:

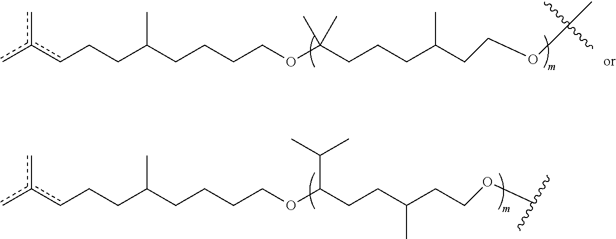

A is selected from substituted or unsubstituted C₁₋₂₀alkyl, substituted or unsubstituted C₂₋₁₂alkenyl, substituted or unsubstituted C₂₋₁₂alkynyl, substituted or unsubstituted C₃₋₁₂cycloalkyl, and substituted or unsubstituted C₆₋₁₀aryl; and each n and m are independently an integer between 0 and 20.

In further embodiments of the third aspect, the disclosure provides any of the following:
3.1 Compound 3, wherein R⁴ is H.
3.2 Compound 3, wherein R⁴ is Si(C₁₋₄ alkyl)₃.
3.3 Compound 3.2, wherein each C₁₋₄alkyl is independently selected from methyl, ethyl, propyl, isopropyl, n-butyl, isobutyl, sec-butyl, and tert-butyl.
3.4 Compound 3.3, wherein each C₁₋₄alkyl is independently selected from methyl and ethyl.
3.5 Compound 3, wherein R⁴ is —C(O)—R⁵.
3.6 Compound 3, wherein R⁴ is —C(O)-A-C(O)—R⁵.
3.7 Compound 3, wherein R⁴ is -A-C(O)—R⁵.
3.8 Compound 3, wherein R⁴ is -A-O—R⁵.
3.9 Compound 3, or any of 3.5-3.8, wherein R⁵ is a moiety:

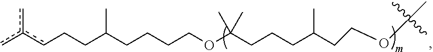

or a moiety:

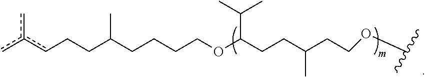

3.10 Compound 3.9, wherein m is an integer between 0 and 10, e.g., between 0 and 8, or between 0 and 6, or between 0 and 4.
3.11 Compound 3.10, wherein m is selected from 0, 1, 2 and 3.
3.12 Compound 3, or any of 3.5-3.8, wherein R⁵ is substituted or unsubstituted C₁₋₂₂alkyl, e.g., substituted or unsubstituted C₁₋₁₆ alkyl, or substituted or unsubstituted C₁₋₁₂alkyl, or substituted or unsubstituted C₁₋₆ alkyl.
3.13 Compound 3.12, wherein R⁵ is selected from unsubstituted methyl, ethyl, propyl, isopropyl, n-butyl, isobutyl, sec-butyl, tert-butyl, n-pentyl, n-hexyl, n-octyl, or n-decyl.
3.14 Compound 3, or any of 3.5-3.8, wherein R⁵ is substituted or unsubstituted C₂₋₂₂alkenyl, e.g., substituted or unsubstituted C₂₋₁₆ alkenyl, or substituted or unsubstituted C₂₋₁₂alkenyl, or substituted or unsubstituted C₂₋₆ alkenyl.
3.15 Compound 3.14, wherein R⁵ is selected from mono-unsaturated and unsubstituted C₇₋₂₀ alkenyl, C₁₀₋₂₀ alkenyl, C₁₂₋₂₀ alkenyl, C₁₄₋₂₀ alkenyl and C₁₆₋₁₈ alkenyl.
3.16 Compound 3.14, wherein R⁵ is selected from di-unsaturated and unsubstituted C₇₋₂₀ alkenyl, C₁₀₋₂₀ alkenyl, C₁₂₋₂₀ alkenyl, C₁₄₋₂₀ alkenyl and C₁₆₋₁₈ alkenyl.
3.17 Compound 3, or any of 3.5-3.8, wherein R⁵ is substituted or unsubstituted C₂₋₂₂alkynyl, e.g., substituted or unsubstituted C₂₋₁₆alkynyl, or substituted or unsubstituted C₂₋₁₂alkynyl, or substituted or unsubstituted C₂₋₆ alkynyl.
3.18 Compound 3, or any of 3.5-3.8, wherein R⁵ is substituted or unsubstituted C₆₋₁₀aryl, e.g., substituted or unsubstituted phenyl or napthyl.
3.19 Compound 3, or any of 3.5-3.8, wherein R⁵ is OH.
3.20 Compound 3 or any of 3.6-3.19, wherein A is substituted or unsubstituted C₁₋₂₀alkyl, e.g., substituted or unsubstituted C₁₋₁₆ alkyl, or substituted or unsubstituted C₁₋₁₂alkyl, or substituted or unsubstituted C₁₋₆ alkyl.
3.21 Compound 2.20, wherein A is (CH₂)_{y} wherein y is an integer from 1 to 6, e.g., wherein y is 2, 3, 4, 5 or 6.
3.22 Compound 3.21, wherein A is (CH₂)₃ or (CH₂)₆.
3.23 Compound 3.21, wherein A is (CH₂)₂.
3.24 Compound 3 or any of 3.1-3.23, wherein n is 0 to 8.
3.25 Compound 3 or any of 3.1-3.23, wherein n 0 to 6.
3.26 Compound 3 or any of 3.1-3.23, wherein n is 0 to 4.
3.27 Compound 3 or any of 3.1-3.23, wherein n is 0, 1, 2, 3, or 4.
3.28 Compound 3 or any of 3.1-3.23, wherein n is 0, 1 or 2.

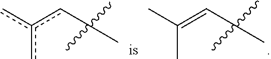

3.29 Compound 3 or any of 3.1-3.28, wherein the terminal group

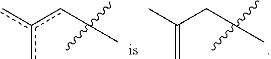

3.30 Compound 3 or any of 3.1-3.28, wherein the terminal group

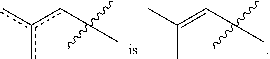

3.31 Compound 3 or any of 3.1-3.28, wherein the terminal group
3.32 Compound 3 or any of 3.1-3.31, wherein the compound of Formula III is enantiomerically enriched, e.g., having a diastereomeric excess (e.g., an enantiomeric excess (e.e.)) of greater than 70%.
3.33 Compound 3.32, wherein the compound of Formula III has a diastereomeric excess (e.g., an enantiomeric excess (e.e.)) of greater than 80%, or greater than 85%, or greater than 90%, or greater than 95%, or greater than 97% or greater than 99%.
3.34 Compound 3.32 or 3.33, wherein each chiral carbon of the compound of Formula III, exclusive of the group R⁴, has the (R)-configuration, optionally wherein each chiral carbon of the group R⁴ also has the (R)-configuration.
3.35 Compound 3.32 or 3.33, wherein each chiral carbon of the compound of Formula III, exclusive of the group R⁴, has the (S)-configuration, optionally wherein each chiral carbon of the group R⁴ also has the (S)-configuration.
3.36 Compound 3.32 or 3.33, wherein each chiral carbon of the compound of Formula III, exclusive of the group R⁴, has the (R)-configuration, and wherein each chiral carbon of the group R⁴ has the (S)-configuration.
3.37 Compound 3.32 or 3.33, wherein each chiral carbon of the compound of Formula III, exclusive of the group R⁴, has the (S)-configuration, and wherein each chiral carbon of the group R⁴ has the (R)-configuration.
3.38 Compound 3, or any of 3.1-3.37, wherein the compound of Formula III is selected from the group consisting of:

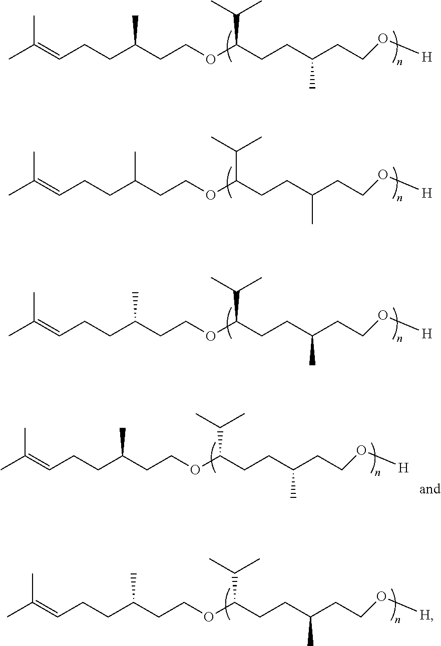

wherein n is an integer from 0 to 10, e.g., 0 to 6, or 0 to 4, or 0, 1, 2 or 3.
3.39 A Composition comprising any one of Compound 3 or any of 3.1-3.38.
3.40 The Composition 3.39, wherein the composition comprises a single compound of Formula III according to Compound 3 or any of 3.1-3.38, wherein the compound is present to an extent of greater than 40%, e.g., greater than 50%, greater than 60%, or greater than 70%, or greater than 80%, or greater than 90%, or 40-70% or 40-60%, or 40-50%, said percent being measured either as the number percent of the molecules in the composition or as the weight percent of the total weight of the composition.
3.41 Composition 3.40, wherein the composition comprises one or more compounds of Formula III (e.g., from one up to ten specific compounds), wherein each compound is independently a compound according to Compound 3 or 3.1-3.38, each compound being present in an amount of at least 1% and up to 90%, e.g., 5 to 50%, or 5 to 40% or 5 to 30%, or 5 to 25%, or 5 to 20% or 5 to 15%, or 5 to 10%, or 40 to 50%, or 30 to 40% or 20 to 30%, or 10 to 20%, or 1 to 10%, or 1 to 5%, or 1 to 2%, said percent being measured either as the number percent of the molecules in the composition or as the weight percent of the total weight of the composition.
3.42 Any one of Compositions 3.41 to 3.41, wherein the number average molecular weight (Mₙ) of the compounds of Formula III in the composition, optionally exclusive of the group R⁴, is 150 to 2000 Daltons.
3.43 Composition 3.42, wherein the number average molecular weight (Mₙ) of the compounds of Formula III in the composition, optionally exclusive of the group R⁴, is 300 to 1900 Daltons, e.g., 300 to 1600 Daltons, or 300 to 1300 Daltons, or 300 to 1100 Daltons, or 300 to 1000 Daltons, or 300 to 800 Daltons, or 300 to 700 Daltons, or 300 to 500 Daltons, or 400 to 1000 Daltons, or 400 to 700 Daltons, or 600 to 1100 Daltons, or 600 to 1000 Daltons, or 600 to 800 Daltons or about 500 Daltons, or about 414 Daltons.
3.44 Any one of Compositions 3.40 to 3.43, wherein the weight average molecular weight (M_{w}) of the compounds of Formula III in the composition, optionally exclusive of the group R⁴, is 150 to 2000 Daltons.
3.45 Composition 3.44, wherein the weight average molecular weight (M_{w}) of the compounds of Formula III in the composition, optionally exclusive of the group R⁴, is 300 to 1900 Daltons, e.g., 300 to 1600 Daltons, or 300 to 1300 Daltons, or 300 to 1100 Daltons, or 300 to 1000 Daltons, or 300 to 800 Daltons, or 300 to 700 Daltons, or 300 to 500 Daltons, or 400 to 1000 Daltons, or 400 to 700 Daltons, or 600 to 1100 Daltons, or 600 to 1000 Daltons, or 600 to 800 Daltons, or about 438 Daltons.
3.46 Any one of Compositions 3.40 to 3.46, wherein the polydispersity (M_{w}/Mₙ) of the compounds of Formula III in the composition (optionally without taking into account the mass of the group R⁴) is in the range of 1 to 5.
3.47 Composition 3.46, wherein the polydispersity (M_{w}/Mₙ) is in the range of 1 to 4, or 1 to 3, or 1 to 2.5, or 1 to 2, or 1 to 1.5, or about 1 to 1.25, or about 1, or 1.5 to 3.5, or 1.5 to 2.5, or about 1.5, or 2 to 4, or 2 to 3, or 2 to 2.5, or about 2, or 1 to 1.25, or 1 to 1.20, or 1 to 1.15, or 1 to 1.10, or about 1.06.
3.48 Any one of Compositions 3.40 to 3.47, wherein the compounds of Formula III in the composition have an average n value of 0 to 8, measured either as a weight average or number average.
3.49 Composition 3.48, wherein the compounds of Formula III in the composition have an average n value of 0 to 6, measured either as a weight average or number average.
3.50 Composition 3.48, wherein the compounds of Formula III in the composition have an average n value of 0 to 5, measured either as a weight average or number average.
3.51 Composition 3.48, wherein the compounds of Formula III in the composition have an average n value of about 0, about 1, about 2, about 3, or about 4, measured either as a weight average or number average.
3.52 Composition 3.48, wherein the compounds of Formula III in the composition have an average n value of about 0, about 1 or about 2, measured either as a weight average or number average.

It is understood that the compounds of Formula III consist of a citronellol polymer backbone that may be formed via a controlled homopolymerization reaction of citronellol to form the compound of Formula III wherein R⁴ is H. In a subsequent reaction (or more than one), a derivative may be formed wherein R⁴ is a moiety other than H. The resulting citronellol homopolymer derivative will necessarily substantially retain the polymeric structural features—for example the values of n and the structural relationships among and between the citronellol monomer units of the polymer backbone—from the previously formed hydroxylic polymer. As a result, when applied to compounds of Formula III wherein R⁴ is a moiety other than H, it is understood that figures for average molecular weight and polydispersity may be described based on the polymer backbone of the molecule excluding the group R⁴. For example, measurements and calculations of molecular weight and polydispersity can be made on the alcoholic polymer (R⁴ is H) prior to further derivatization, and the resulting values can be extrapolated to later-derived derivatives (wherein R⁴ is a moiety other than H).

The term “compounds of the present disclosure” and “compositions of the present disclosure” include the compounds of Formula I, Formula II and Formula III, and the compounds and compositions according to Formulas 1.1-1.68, 2.1-2.51, and 3.1-3.52, and 1.69-1.77, and any and all other embodiments thereof. The compounds of the present disclosure each consist of a homopolymeric polyether backbone with a terminal hydroxy group, wherein the terminal hydroxy group is optionally further functionalized to form an ether, ester or similar linkage. For example, in the compounds of Formula I, the polyether backbone is that portion of the compound excluding the group R². In the compounds of Formula II and Formula III the polyether backbone is that portion of the compound excluding the groups R⁴.

In some embodiments of the present disclosure, the compounds and compositions of the present disclosure have a polyether backbone having a number average molar weight (M_{w}) of in the range of from 150-3000 g/mol, preferably in the range of from 00-500 g/mol, for example, as measured by means of isocratic chromatography using THF as a mobile phase in HPLC.

In some embodiments, the compositions of the present disclosure have a polydispersity (M_{w}/Mₙ) in the range of 1 to 5, and preferably 1 to 2. In some embodiments, the compounds of the present disclosure are based on a polyether alcohol derived from the homopolymerization product of citronellol, geraniol, linalool, citronellic acid, limonene, dihydromyrcene, myrcenol, adipic acid, propanediol, ethylene glycol, glycerol, 1,9-nonnanediol or 1,6-hexanediol. Preferably they are based on the polymerization of citronellol and derivatization thereof.

In some embodiments, the compositions of the present disclosure comprise compounds having number average molecular weight of polyether alcohol and derivatives thereof in the range of from 100-1000 g/mol, preferably in the range from 100-500 g/mol. Number average molecular weight may be measured by means of isocratic chromatography, such as, using Agilent Oligopore GPC column and THF as a mobile phase in HPLC.

Compounds and compositions of the present disclosure, especially those having a polydispersity of 1 to 2, have one or more of the following favorable features: the compounds are short-chain polymers; the compounds are made using a reversible polymerization reaction; the polymers are biodegradable and biocompatible; and the polymers may be manufactured using all-natural ingredients. These are important benefits in many of the commercial applications in which these compounds may be used. The compositions disclosed herein, with the polydispersity in the range of from 1 to 5, preferably 1 to 2, are suitable as replacement or substitutes for surfactants, polymers, and silicones in a variety of commercial products, such as in cosmetics and pharmaceutical compositions, and as adjuvants in crop care formulations, and as lubricants or solvents in enhanced oil recovery, fracking and oil field applications. The compounds and compositions disclosed herein offer improved physical characteristics, such as appearance, odor, viscosity, refractive index and/or surface tension.

In some embodiments, the compositions of the present disclosure comprise compounds having a weight average molecular weight (M_{w}) of equal to or greater than 100, 200, 300, 400, 500, 600, 700, 800, 900 and 1000 Daltons. In another embodiment, the weight average molecular weight may be selected from the group consisting of: 330, 331, 332, 333, 334, 335, 336, 337, 338, 339, 340, 341, 342, 343, 344, 345, 346, 347, 348, 349, 350, 351, 352, 353, 354, 355, 356, 357, 358, 359 and 360 Daltons.

In some embodiments of this invention, the polydispersity index (PDI) of the compounds and compositions of the disclosure is less than or about 1.25, 1.24, 1.23, 1.22, 1.21, 1.20, 1.19, 1.18, 1.17, 1.16, 1.15, 1.14, 1.13, 1.12, 1.11 or 1.10. In some embodiments, the PDI may be equal to or greater than 1.00, 1.01, 1.02, 1.03, 1.04, 1.05, 1.06, 1.07, 1.08 or 1.09.

In some embodiments of the present disclosure, the compounds and compositions disclosed herein are useful: for fragrance retention, fixation of fragrances, or as a fragrance carrier; as a malodor counteracting agent; in paints and coatings; as an adjuvant for crop control; as a cosmetic ingredient (e.g., as a silicone replacement or a white oil replacement); in nail polish; in writing or printing inks; as a resin or resin-replacement; as an insect repellant (e.g., a mosquito repellent); and in sun block formulations. The polymeric compounds with aforementioned properties can have one of the following average molecular weight (M_{w}) 390, 391, 392, 393, 394, 395, 396, 397, 398, 399, 400, 401, 402, 403, 404, 405, 406, 407, 408, 409, 410, 411, 412, 413, 414, 415, 416, 417, 418, 419 and 420 Daltons. In other embodiments the polymeric compounds with aforementioned properties can have polydispersity index (PDI), equal or greater than 1.00, 1.01, 1.02, 1.03, 1.04, 1.05, 1.06, 1.07, 1.08 and 1.09.

Methods to make these ethers similar those described herein are described in US2017/0283553 and WO2019/028053, the contents of which are incorporated by reference herein in their entireties. Such polymers can generally be made with high degrees of polymerization in a short period of time by using a resin-bound acid catalyst, such as Amberlyst®, under neat, solvent-free conditions. Amberlyst-type resins are recognized in the art and understood to be macroreticular or cellular resin covalently bonded to sulfonic acid or carboxylic acid groups, preferably sulfonic acid groups. Such polymerizations can be done at or below room temperature, preferably at slightly elevated temperature, between 30 and 110° C., or even more preferably between 40 and 90° C. (e.g., about 50° C.). Such polymerizations can take place in batch reactors, semi-batch reactors, or even more preferably in continuous packed bed-type reactors of the type described in U.S. Provisional Application 62/384,939 and PCT/US2017/50808, the contents of each of which are incorporated herein by reference.

The reversibility of the polymerization of the claimed compounds derives from the nature of these polymers, having adjacent oxygen atoms and tertiary carbon atoms. As a result, under conditions which will promote the cleavage of the O—C bond, the resulting tertiary carbocation is unusually stable. This leads to facile abstraction of an adjacent hydrogen atom to regenerate the starting materials' alcohol and alkene functional groups. Such de-polymerization may be promoted by mildly acidic conditions (e.g., with Lewis acids or Bronsted acids) or by thermal conditions or by enzymatic conditions (as by enzymes found in naturally occurring bacteria).

This depolymerization property results in biodegradation. This property also permits the formation of compositions comprising the compounds wherein the depolymerization of the polymers may be controlled to permit slow release of the monomeric polymer constituents (e.g., citronellol) or of shortened polymeric constituents (e.g., the release of dimers of citronellol by depolymerization of a larger polymer). The present disclosure embraces solid and/or liquid formulations (e.g., products) comprising any of the compounds or composition described herein, such that the wherein the formulation provides for slow, controlled depolymerization of the polymers and diffusion of the resulting monomers and or shortened oligomers so that that can be released from the composition (e.g., by vaporization at the surface of the composition). Such formulations may be comprised of ingredients which accelerate such depolymerization (such as Lewis acids or Bronsted acids, or enzymes) or such formulations may be associated with a device comprising an electrical heating element to promote thermal depolymerization. The monomers and/or shortened oligomers produced in this manner (e.g., citronellol or dimers or trimers of citronellol) are themselves beneficial for any number of reasons, e.g., as fragrances, insect repellants, anti-oxidants, anti-microbials, or as active pharmaceutical ingredients (e.g., where the composition is a pharmaceutical composition).

The compounds and compositions disclosed herein are particularly suitable for the replacement of silicones, mineral oil and/or paraffins, in cosmetic compositions, such as concealers, primers and/or moisturizers.

In a fourth aspect, the present disclosure provides a product composition (Product 1), which product composition comprises a Compound 1, a Compound 2 or any of 2.1-2.37, or a Compound 3 or any of 3.1-3.38, or Composition 1 or any of 1.1-1.77, ora Composition 2.38-2.51, or a Composition 3.39-3.52, or any mixtures thereof, in combination with at least one suitable solvent, carrier, or other excipient. In further embodiments of the fourth aspect, the present disclosure provides product compositions as follows:
1.1 A fragrance product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.2 A perfume product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.3 A soap product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.4 An insect repellant product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof, for example, a mosquito repellant product composition.
1.5 An insecticide product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.6 A detergent product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.7 A household cleaning agent product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.8 An air freshener product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.9 A room spray product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.10 A pomander product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.11 A candle product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.12 Product composition 1.11, wherein the product composition further comprises a paraffin wax and/or beeswax base.
1.13 Product composition 1.12, wherein the product composition consists of the Compound 2 or any of 2.1-2.37, or the Compound 3 or any of 3.1-3.38, or the Composition 1 or any of 1.1-1.68, or the Composition 2.38-2.51, or the Composition 3.39-3.52, or any mixtures thereof, dispersed within the paraffin wax and/or beeswax base, with a suitable wick embedded therein.
1.14 A cosmetic product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.15 A toilet water product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.16 A pre- and aftershave lotion product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.17 A talcum powder product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.18 A hair-care product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.19 A body deodorant product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.20 An anti-perspirant product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.21 A shampoo product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.22 A pet litter product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.23 A topically applied skin care product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof, optionally wherein the skin care product is selected from skin-conditioning agents; skin penetration enhancing agents; skin protectants; skin soothing agents; shaving creams and shaving gels; skin creams and lotions (e.g., moisturizing creams and lotions); skin healing agents; ultraviolet light absorbers or scattering agents; sequestrants; anti-acne agents; anti-androgens; depilation agents; keratolytic agents/desquamation agents/exfoliants such as salicylic acid; panthenol moisturizer such as D-panthenol; soluble or colloidally-soluble moisturizing agents such as hyaluronic acid and starch-grafted sodium polyacrylates; and sunscreens.
1.24 Product composition 1.23, wherein the skin care product is a skin protectant.
1.25 Product composition 1.23, wherein the skin care product is a skin soothing agent.
1.26 Product composition 1.23, wherein the skin care product is a sunscreen.
1.27 A paint or coating product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.28 A lubricant product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.29 A plastic product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.30 A defoamer product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.31 A hydraulic fluid product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.32 An antimicrobial product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.33 A crop care product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof, e.g., wherein said compound is an adjuvant in the crop care formulation.
1.34 A product composition for enhanced oil recovery, fracking and/or other oil field applications comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof, e.g., wherein said compound is lubricant or solvent in said formulation.
1.35 A product comprising any preceding composition wherein said composition is stored or housed in a receptacle comprising an electrical heating element, wherein actuation of the heating element results in heating of the composition, thermal decomposition of the Compound, and release of volatile substances.
1.36 A fragrance of perfume product comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof, wherein said compound is used as a fragrance retention agent, a fragrance fixative or a fragrance carrier.
1.37 A cosmetic product a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof, wherein said compound is used as a replacement for silicone and/or as a replacement for white oil.
1.38 A nail polish product comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.39 A writing ink or printing ink product comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.40 An adhesive product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof.
1.41 An oral care product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof; optionally wherein the oral care product composition is selected from a toothpaste, tooth gel, or mouthwash.
1.42 A food product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof; optionally wherein the food product composition is selected from a chewing gum or carbonate beverage.
1.43 A pharmaceutical product composition comprising a Compound 2 or any of 2.1-2.37, or a Compound 3 or any of 3.1-3.38, or Composition 1 or any of 1.1-1.68, or a Composition 2.38-2.51, or a Composition 3.39-3.52, or any mixtures thereof; optionally wherein the pharmaceutical product composition is selected from a capsule, a tablet (e.g., a chewable tablet), a syrup (e.g., a cough syrup), a lozenge (e.g., a cough drop), and a liquid (e.g., a solution or suspension for oral ingestion).
1.44 Product 1, or any of 1.1-1.43, wherein the product composition further comprises one or more hydrocarbons.
1.45 Product composition 1.44, wherein the hydrocarbons comprise linear or branched aliphatic hydrocarbons having from 7 to 30 carbon atoms, e.g., 7 to 25 carbon atoms, or 7 to 23 carbon atoms, or 7 to 12 carbon atoms, or 12 to 15 carbon atoms, or 15 to 18 carbon atoms, or 18 to 21 carbon atoms, or 21 to 23 carbon atoms, or 23 to 28 carbon atoms, or any combination thereof.
1.46 Product composition 1.44, wherein the hydrocarbons comprise branched or unbranched cyclic aliphatic hydrocarbons having from 5 to 20 carbon atoms, e.g., 5 to 16 carbon atoms, or 5 to 12 carbon atoms, or 5 to 10 carbon atoms, or 5 to 8 carbon atoms, or 8 to 10 carbon atoms, or 10 to 12 carbon atoms, or 12 to 16 carbon atoms, or 16 to 20 carbon atoms, or any combination thereof.
1.47 Any one of Product compositions 1.44-1.46, wherein the hydrocarbons comprise saturated hydrocarbons, monounsaturated hydrocarbons, polyunsaturated hydrocarbons, or some combination thereof.
1.48 Any one of Product compositions 1.44-1.47, wherein the hydrocarbons do not comprise aromatic hydrocarbons, and/or do not comprise cyclic hydrocarbons, e.g., wherein the product composition does not comprise aromatic hydrocarbons and/or cyclic hydrocarbons.
1.49 Any one of Product compositions 1.44-1.48, wherein the one or more hydrocarbons are selected from heptane, octane, nonane, decane, undecane, dodecane, isododecane, tridecane, tetradecane, pentadecane, hexadecane, heptadecane, octadecane, nonadecane, henicosane, docosane, and tricosane, and any saturated linear or saturated branched isomer thereof.
1.50 Product composition 1.49, wherein the one or more hydrocarbons are selected from undecane, tridecane, or a mixture thereof.
1.51 Any of Product compositions 1.44-1.50, wherein the one or more hydrocarbons comprises a net amount of 0.1 to 95% by weight of the product composition, e.g., 1 to 90%, or 5 to 80%, or 10 to 70%, or 15 to 60%, or 5 to 60%, or 10 to 50%, or 15 to 40%, or 1 to 30%, or 1 to 25%, or 5 to 20%, or 20 to 40%, or 40 to 60%, or 60 to 80%, or 80-95%, or 25 to 75%, or about 50%, by weight of the product composition.
1.52 Any of Product compositions 1.44-1.51, wherein the product composition consists of, or consists essentially of, from 5 to 95% by weight of a Compound 2 or any of 2.1-2.37, or a Compound 3 or any of 3.1-3.38, or Composition 1 or any of 1.1-1.68, and the balance (from 95 to 5%) by weight of the hydrocarbon mixture described in any of embodiments 1.44-1.51.
1.53 Any preceding Product composition, wherein the product composition is an aerosol product.
1.54 Product composition 1.53, wherein the product is an aerosol deodorant, aerosol anti-perspirant, aerosol body spray, aerosol air freshener, aerosol fragrance or perfume product, aerosol insecticide, aerosol lubricant, aerosol hair care product (e.g., hair spray), aerosol sunscreen, aerosol throat analgesic, or aerosol insect repellant.
1.55 Product 1.53 or 1.54 wherein the Product composition further comprises an aerosol propellant, e.g., propane, n-butane, isobutane, dimethyl ether, diethyl ether, methyl ethyl ether, chlorofluorocarbons, fluorocarbons, hydrochlorofluorocarbons, hydrofluorocarbons (e.g., 1,1-difluoroethane, or 1,1,1,-trifluoroethane, or 1,1,1,2-tetrafluoroethane, or 1,1,1,3,3-pentafluoropropane, or 1,1,1,3,3-pentafluorobutane, or 1,1,1,2,3,3,3-heptafluoropropane, or 1,3,3,3-tetrafluoropropene, or 2,3,3,3-tetrafluoropropene), nitrous oxide, carbon dioxide, nitrogen, air or any combination thereof.
1.56 Any of Product compositions 1.53-1.55, wherein the product is an aerosol body spray, optionally comprising a propellant selected from propane, n-butane, isobutane, 1,1-difluoroethane, 1,1,1-trifluoroethane, 1,1,1,2-tetrafluoroethane, 1,1,1,3,3-pentafluoropropane, 1,1,1,3,3-pentafluorobutane, 1,1,1,2,3,3,3-heptafluoropropane, and a combination thereof, such as in particular, a combination of propane, n-butane, isobutane and 1,1-difluoroethane.
1.57 Any preceding Product, wherein the product comprises arrowroot powder (e.g., as an alternative or substitute for talc).

In another embodiment, the present disclosure provides a Compound 1, or a Compound 2 or any of 2.1-2.37, or a Compound 3 or any of 3.1-3.38, or Composition 1 or any of 1.1-1.77, or a Composition 2.38-2.51, or a Composition 3.39-3.52, or any mixtures thereof, for use in Product Composition 1, or any of 1.1-1.57.

In other embodiments of the preceding aspects, the present disclosure provides a composition comprising Compound 1, Compound 2 or any of 2.1-2.37, or Compound 3 or any of 3.1-3.38, or Composition 1 or any of 1.1-1.77, or Composition 2 or any of 2.38-2.51, or Composition 3 or any of 3.39-3.52, or Product 1 or any of 1.1-1.57, wherein the composition or product further comprises one or more additives selected from: a cooling sensate, a warming sensate and/or a tingling sensate; a flavorant or fragrance; vitamins, minerals, nutraceuticals, energizing agents, soothing agents, coloring agents, amino acids antioxidants, preservatives, pH modifying agents, viscosity adjusting agents, emulsifiers, and combinations of any preceding.

In particular embodiments, such compositions comprise a cooling, warming and/or tingling sensate. Such compositions may enhance or reduce the impact of the sensate, such as by dilution, or may attenuate or otherwise alter the properties or perception of the sensate, due to antagonistic or synergistic effects between the components of the compositions. Sensates are useful to impart cooling, warming, and/or tingling sensations to the skin or to mucous membranes of the oral cavity or pharynx. As such, sensates may be useful as flavors or fragrances in a wide range of compositions and products. Compositions such as these may provide an immediate warming, cooling, and or tingling sensation upon application of the composition to the body. In some embodiments, this helps provide an emollient effect. In some embodiments, the compounds of the present disclosure (e.g., Compound 1, 2 and/or 3) may also serve as a viscosity adjusting agent, a carrier, a distributing agent, a diluent agent, or a retention agent, for the sensate in the composition. This may help result in a sensate composition which imparts a controlled, sustained and/or delayed cooling, warming or tingling sensation.

In some embodiments of the above aspect, the sensate interacts with the TRPV protein in order to induce or reduce the desired cooling or warming effect. In some embodiments of the above aspect, the sensate is a natural compound, and in other embodiments the sensate is a synthetic compound.

Suitable cooling sensates include: menthol, levomenthol, peppermint oil, n-N-substituted-p-menthane-3-carboxamides, acyclic tertiary and secondary carboxamides, 3-1-menthoxy propane-1,2-diol, and mixtures thereof.

Suitable warming sensates include: vanillyl n-butyl ether, vanillyl alcohol n-propyl ether, vanillyl alcohol isopropyl ether, vanillyl alcohol isobutyl ether, vanillyl alcohol n-amino ether, vanillyl alcohol isoamyl ether, vanillyl alcohol n-hexyl ether, vanillyl alcohol methyl ether, vanillyl alcohol ethyl ether, gingerol, shogaol, paradol, zingerone, capsaicin, dihydrocapsaicin, nordihydrocapsaicin, homocapsaicin, homodihydrocapsaicin, ethanol, isopropyl alcohol, isoamyl alcohol, benzyl alcohol, eugenol, cinnamon oil, cinnamic aldehyde and phosphate derivatives thereof, pine needle oil, wintergreen oil, rosemary oil, eucalyptus oil, incense oil, and mixtures thereof. In some embodiments, the cooling sensate is combined with a metal ion (e.g., stannous, calcium, zinc, copper and the like) or a non-metal counter ion (e.g., fluoride and the like) to provide enhanced activity of a coolant in term of onset, intensity, or impact and duration.

Suitable tingling sensates include Jambu oleoresin extract, particularly for use in food products.

In some embodiments, a sensate is used to induce a revulsive effect, particularly in topical compositions. Suitable sensates for this purpose include menthol, pine needle oil, orange oil, lemon oil, wintergreen oil, bergamot oil, rosemary oil, lavender oil, glycosaminoglycans, and mixtures thereof.

In some embodiments, the sensate is a chemesthetic compound, which is a compound which induces trigeminal sensation.

Product compositions according to the present disclosure comprising such sensates include: cosmetics (such as lipstick, after shave lotions, foundations and the like), personal care products (such as skin creams, astringent lotions, cleansing lotions, deodorants, shampoos, conditioners, soaps, hair gels, hair tonics, hair growth stimulants, shaving foams, shaving creams, bubbling bath beads, insect repellent sprays, and the like) and pharmaceuticals products (such as, analgesic preparations, lozenges and the like).

In some embodiments, the compositions described herein may comprise one or more emulsifiers or a blend of emulsifiers, preferably emulsifiers approved as food and/or cosmetic ingredients. Such emulsifiers may include any one or more of: fatty acid lactylate esters or esters salts, fatty acids, fatty alcohols, fatty acid monoglycerides, fatty acid diglycerides, lecithin, soy lecithin, and celluloses. Suitable emulsifiers include: palmitoyl lactylate and salts thereof, stearoyl lactylate and salts thereof, myristoyl lactylate and salts thereof, lauryl lactylate and salts thereof, arachidonoyl laurylate and salts thereof, behenyl lactylate and salts thereof, lauric acid, palmitic acid, stearic acid, behenic acid, myristic acid, arachidonic acid, lauryl alcohol, myristyl alcohol, palmityl alcohol, stearyl alcohol, arachidonoyl alcohol, behenyl alcohol, glycerol monostearate, glycerol monopalmitate, glycerol monolaurate, glycerol monomyristate, glycerol monobehenate, and glycerol monoarachidonate. Salts of the fatty acid lactylate esters may be sodium, potassium, lithium, calcium or magnesium salts. In some embodiments, the compositions of the present disclosure include an “emulsification blend.” As used, herein, the term “emulsification blend” refers to an emulsifier mixture comprising a (1) a fatty acid lactylate ester salt (e.g., a salt of palmitoyl lactylate, stearoyl lactylate, myristoyl lactylate, lauryl lactylate, or behenyl lactylate), (2) a fatty alcohol (e.g., lauryl alcohol, myristyl alcohol, palmityl alcohol, stearyl alcohol, or behenyl alcohol), and (3) a fatty acid monoglyceride (e.g., glycerol monostearate, glycerol monopalmitate, glycerol monolaurate, glycerol monomyristate, glycerol monobehenate).

Other suitable additives include: flavorants (e.g., berry flavors such as pomegranate, acai, raspberry, blueberry, strawberry, boysenberry, and/or cranberry; natural or synthetic flavors or aromas, such as peppermint, spearmint, wintergreen, chocolate, licorice, citrus; fruit flavors, such as apple, peach, pear, cherry, plum, orange, lime, grape, mango, passion fruit, pineapple, and grapefruit, gamma octalactone, vanillin, ethyl vanilline, butter, rum, coconut, almond, pecan, walnut, hazelnut, French vanilla, sugar cane, maple, cassis, caramel, banana, malt, espresso, white chocolate, spice flavors such as cinnamon, clove, cilantro, basil, oregano, garlic, mustard, nutmeg, rosemary, thyme, tarragon, dill, sage, anise, and fennel, methyl salicylate, linalool, jasmine, coffee, olive oil, sesame oil, sunflower oil, bergamot oil, geranium oil, peanut oil, lemon oil, ginger oil, balsamic vinegar, rice wine vinegar, red wine vinegar; vegetable flavors, such as tomato, carrot, spinach, broccoli, squash, onion, beet, turnip, parsnip, asparagus, pepper, fennel, zucchini, potato; and any combinations thereof); supplemental vitamins (e.g., vitamin A, vitamin D, vitamin E, vitamin K, thiamine, riboflavin, niacin, folic acid, pyridoxin, choline, inositol, vitamin B12, PABA, vitamin C, and mixtures thereof); preservatives (e.g., methyl paraben, propyl paraben, sodium propionate, citric acid, ascorbic acids, sorbic acid alkali metal salts, such as potassium sorbate, benzoic acid alkali metal salts, such as sodium benzoate, and the like); nutraceuticals (e.g., compounds derived from natural food sources and/or genetically modified food sources); amino acids (e.g., valine, leucine, isoleucine, lysine, threonine, tryptophan, methionine, and phenylalanine, alanine, arginine, asparagine, aspartic acid, cysteine, glutamic acid, glutamine, glycine, histidine, proline, serine, tyrosine and mixtures thereof); cyclodextrins (e.g., water soluble cyclodextrins); and cannabinoids, such as cannabidiol (CBD) or delta-9-tetrahydrocannabinol (THC).

In some embodiments described herein, the composition comprises one or more hydrocarbons. The hydrocarbon, or mixture of hydrocarbons, may act as an emollient base, surfactant base, or emulsifier base, or as fragrance fixer, carrier, diluent or thickener. The hydrocarbons in that capacity may help provide a suitable environment for dissolution of and homogenization of the emollient, surfactant and/or emulsifier in the composition. Preferably such compositions comprise from 0.1-30 wt % of the hydrocarbon base. Such compositions may comprise from 0.1 to 30% by weight each of cosmetically acceptable thickener, surfactants, emulsifiers, emollients, preservative, stabilizers, fragrances, or cosmetic actives. Another particularly useful additive is arrowroot powder, which can be used as a substitute for talc, such as in cosmetic compositions. In some embodiments, the present disclosure provides a blend of arrowroot powder and a polymer or polymer mixture as described herein (e.g., Compound 2 or any of 2.1-2.37, or a Compound 3 or any of 3.1-3.38, or Composition 1 or any of 1.1-1.77, or a Composition 2.38-2.51, or a Composition 3.39-3.52) in a ratio of about 95% arrowroot powder and 5% polymer or polymer mixture. In some embodiments any of Product Composition 1, or any of 1.1-1.57, may comprise this arrowroot powder blend.

Compositions according to the present disclosure which include a polymer or polymer mixture (e.g., Compound 2 or any of 2.1-2.37, or a Compound 3 or any of 3.1-3.38, or Composition 1 or any of 1.1-1.68, or a Composition 2.38-2.51, or a Composition 3.39-3.52) in combination with a hydrocarbon may be particularly suited as alternatives for cosmetic and personal care compositions based on a silicone polymer base or mineral oil base. Such compositions provide a thin, smooth skin feel and quick-drying properties. The hydrocarbons also improve the spreadability, skin breathability and lubricating properties of the compositions. Such compositions are also non-comedogenic (they do not clog skin pores) and non-occlusive.

Compositions such as these are particularly useful as an natural alternative to Vaseline or petroleum ointment, or as a balm, where the polymers and mixture of hydrocarbons can be used in combination with natural occlusive ingredient such as sunflower seed oil, jojoba butter, avocado oil, jojoba seed oil, grape seed oil, coconut oil, hydrogenated vegetable oil, kukuinut seed oil, shea butter, hemp seed oil, hydrogenated grape seed oil, meadowfoam seed oil, mango seed butter, rice bran seed oil, rosehip fruit oil, soy lecithin, cupuacu seed butter, pumpkin seed oil, chamomile flower extract, bergamot fruit oil, palmarosa oil, lavender oil, beeswax, rosemary extract, clary sage oil, cocoa butter, soybean oil, calendula flower extract, jasmine absolute, gardenia flowers to nourish and moist skin without being super greasy. This type of natural personal care product can be used on hands, feet, body and even as a gentle makeup remover.

Composition 1 or any of 1.1-1.77, or a Composition 2.38-2.51, or a Composition 3.39-3.52, or any mixtures thereof, for use in Product Composition 1, or any of 1.1-1.57, may further comprise essential oils, such as peppermint oil, lavender oil, sandalwood oil, bergamot oil, rose oil, chamomile oil, ylang-ylang oil, tea-tree oil, jasmine oil, lemon oil, clementine oil, coriander seed oil, corn mint oil, eucalyptus lemon oil, geranium oil, ginger oil, key lime oil, basil oil.

Composition 1 or any of 1.1-1.77, or a Composition 2.38-2.51, or a Composition 3.39-3.52, or any mixtures thereof, for use in Product Composition 1, or any of 1.1-1.57, may further comprise hydrotropes (e.g., renewable hydrotropes). The term “hydrotropes” refers to compounds capable of solubilizing hydrophobic polymers and hydrocarbons in aqueous solution by means other than micellar solubilization.

Composition 1 or any of 1.1-1.77, or a Composition 2.38-2.51, or a Composition 3.39-3.52, or any mixtures thereof, for use in Product Composition 1, or any of 1.1-1.57, may further comprise meroterpenes, such as bakuchiol. Meroterpenes are compounds with a partial terpenoid structure.

In some embodiments, the compositions and products disclosed herein are gluten-free cosmetic or personal care compositions, such as toothpastes, mouthwashes, lipsticks, and lip balms.

In some embodiments, the compositions and products disclosed herein are emulsions, such as water-in-oil emulsions or oil-in-water emulsions.

In other embodiments of the preceding aspects, especially the fourth aspect, the present disclosure provides a composition comprising Compound 1, Compound 2 or any of 2.1-2.37, or Compound 3 or any of 3.1-3.38, or Composition 1 or any of 1.1-1.77, or Composition 2 or any of 2.38-2.51, or Composition 3 or any of 3.39-3.52, or Product 1 or any of 1.1-1.57, optionally wherein the composition or product further comprises one or more additives, hydrocarbons, essential oils, hydrotropes, meroterpenes, as described hereinabove, wherein the composition or product is packaged in a container or device comprising packaging made from high density polyethylene (HDPE), low density polyethylene (LDPE), polypropylene (PP) or polyethylene terephthalate (PET), or a combination thereof. Preferably, the composition or product is packaged in a container or device comprising packaging made from high density polyethylene (HDPE). In further embodiments, the present disclosure provides:
4.1 A composition comprising Compound 1, Compound 2 or any of 2.1-2.37, or Compound 3 or any of 3.1-3.38, or Composition 1 or any of 1.1-1.77, or Composition 2 or any of 2.38-2.51, or Composition 3 or any of 3.39-3.52, or Product 1 or any of 1.1-1.57, optionally wherein the composition or product further comprises one or more additives, hydrocarbons, essential oils, hydrotropes, or meroterpenes, as described hereinabove, wherein the composition or product is packaged in a container or device comprising packaging made from high density polyethylene (HDPE), low density polyethylene (LDPE), polypropylene (PP) or polyethylene terephthalate (PET), or a combination thereof.
4.2 Composition or product 4.1, wherein the packaging comprises or consists of HDPE.
4.3 Composition or product 4.1 or 4.2, wherein the composition or product is a fragrance composition, perfume, soap, insect repellant and insecticide, detergent, household cleaning agent, air freshener, room spray, pomander, candle, cosmetic, toilet water, pre- and aftershave lotion, talcum powder, hair-care product, body deodorant, anti-perspirant, shampoo, skin care applications, pharmaceuticals, antimicrobials, pet litter, crop care formulation, or oil field, fracking or enhanced oil recovery formation.
4.4 Composition or product 4.1, 4.2, or 4.3, wherein the composition or product comprises the following compound:

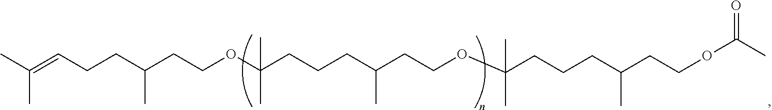

wherein n is an integer from 0-10 (e.g., 0-4, 1-7, or 0, 1, 2, 3, 4, 5, 6, or 7), or a mixture of one or more of such compounds each having a value of integer n from 0-10 (e.g., 0-4, 0-7, or 0, 1, 2, 3, 4, 5, 6 or 7).
4.5 Composition or product 4.1, 4.2, or 4.3, wherein the composition or product comprises the compound having the INCI name “Polycitronellol Acetate,” wherein INCI refers to the International Nomenclature of Cosmetic Ingredients.
4.6 Composition or product 4.4 or 4.5, wherein the composition or product is a cosmetic composition or a cosmetic product.
4.7 The composition or product according to 4.6, wherein the composition or product comprises said compound in a cosmetic preparation which is in the form of an emulsion.
4.8 The composition or product according to 4.7, wherein the emulsion is an oil-in-water emulsion (o/w emulsion).
4.9 The composition or product according to 4.7, wherein the emulsion is a water-in-oil emulsion (w/o emulsion).
4.10 The composition or product according to 4.7, 4.8 or 4.9, wherein the cosmetic preparation comprises said compound in an amount of 0.1 to 10% by weight based on the weight of the cosmetic preparation.
4.11 The composition or product according to any one of 4.4 to 4.10, wherein the composition or product comprises said compound in an amount of 0.1 to 10% by weight based on the weight of the cosmetic composition or product.

In a fifth aspect the, the present disclosure provides a method (Method 1) of using a Compound 2 or any of 2.1-2.37, or a Compound 3 or any of 3.1-3.38, or Composition 1 or any of 1.1-1.77, or a Composition 2.38-2.51, or a Composition 3.39-3.52, or any mixtures thereof, in the manufacture of a Product Composition (e.g., Product Composition 1, et seq.) or Composition or product 4.1-4.11, for example, a fragrance composition, perfume, soap, insect repellant and insecticide, detergent, household cleaning agent, air freshener, room spray, pomander, candle, cosmetic, toilet water, pre- and aftershave lotion, talcum powder, hair-care product, body deodorant, anti-perspirant, shampoo, skin care applications, pharmaceuticals, antimicrobials, pet litter, crop care formulation, or oil field, fracking or enhanced oil recovery formation).

An added benefit of these materials described herein is that they are expected to be fully biodegradable and biocompatible.

During the course of the evaluation of these polyethers, it was surprisingly observed that the depolymerization back to monomer would spontaneously occur at ˜180° C. for the citronellol-type polymers. This thermal depolymerization property, or similar enzymatic and/or, acid catalyzed depolymerization properties could be beneficially used to deliver citronellol monomer in a controlled fashion over time.

In one aspect, thermal depolymerization could be used to deliver monomer into the air in a controlled release. In one aspect, the invention contemplates using the compounds of Compound 1, et seq., and/or Compound 2, et seq., e.g., as produced by Method 1, et seq., e.g., in candles or thermal dispensers used for odor control and/or mosquito control, in low pH industrial cleaners which could have the depolymerized monomer ingredient released over time to promote beneficial odor, and laundry detergents that could use enzymes to digest the polymers over time to have fresh odor over longer periods.

Other aspects regarding the use of compounds and compositions of the present disclosure may be found as disclosed in US2017/0283553 and WO2019/028053, the contents of which are incorporated by reference herein in their entireties

The details of one or more embodiments of the invention are set forth in the accompanying description below. Unless defined otherwise, all technical and scientific terms used herein have the same meaning as commonly understood by one of ordinary skill in the art to which this invention belongs. In the case of conflict, the present specification will control.

Unless otherwise indicated, it is to be understood that the terminology used herein is for the purpose of describing particular embodiments only and is not intended to be limiting. In this specification and in the claims that follow, reference will be made to a number of terms, which shall be defined to have the definitions set forth below.

As used herein, the singular forms “a,” “an,” and “the” include plural referents unless the context clearly dictates otherwise. Thus, for example, reference to “a reactant” includes not only a single reactant but also a combination or mixture of two or more different reactant, reference to “a substituent” includes a single substituent as well as two or more substituents, and the like.

As used herein, the phrases “for example,” “for instance,” “such as,” or “including” are meant to introduce examples that further clarify more general subject matter. These examples are provided only as an aid for understanding the disclosure, and are not meant to be limiting in any fashion. Furthermore, as used herein, the terms “may,” “optional,” “optionally,” or “may optionally” mean that the subsequently described circumstance may or may not occur, so that the description includes instances where the circumstance occurs and instances where it does not. For example, the phrase “optionally present” means that an object may or may not be present, and, thus, the description includes instances wherein the object is present and instances wherein the object is not present.

As used herein, the phrase “having the formula” or “having the structure” is not intended to be limiting and is used in the same way that the term “comprising” is commonly used.

In some formulae of the present application, one or more chiral centers are identified by an asterisk placed next to the chiral carbon. In other formulae, no chiral center is identified, but the chiral isomers are nonetheless covered by these formulae.

Some compounds of the present invention can exist in a tautomeric form which is also intended to be encompassed within the scope of the present invention.

“Tautomers” refers to compounds whose structures differ markedly in arrangement of atoms, but which exist in easy and rapid equilibrium. It is to be understood that the compounds of the invention may be depicted as different tautomers. it should also be understood that when compounds have tautomeric forms, ail tautomeric forms are intended to be within the scope of the invention, and the naming of the compounds does not exclude any tautomeric form. Further, even though one tautomer may be described, the present invention includes all tautomers of the present compounds.

As used herein, the term “salt” can include acid addition salts including hydrochlorides, hydrobromides, phosphates, sulfates, hydrogen sulfates, alkylsulfonates, arylsulfonates, acetates, benzoates, citrates, maleates, fumarates, succinates, lactates, and tartrates; alkali metal cations such as Na⁺, K⁺, Li₊, alkali earth metal salts such as Mg²⁺ or Ca²⁺, or organic amine salts, or organic phosphonium salts.

The term “alkyl” as used herein refers to a monovalent or bivalent, branched or unbranched saturated hydrocarbon group having from 1 to 22 carbon atoms, typically although, not necessarily, containing 1 to about 12 carbon atoms, such as methyl, ethyl, n-propyl, isopropyl, n-butyl, isobutyl, t-butyl, octyl, and the like.

As used herein throughout, the term “unsaturated alkyl” is understood as being the same as “alkenyl.” Thus, the term “optionally unsaturated alkyl ester” refers to an ester having either an alkyl chain or an alkenyl chain. Thus, in this context, “optionally unsaturated alkyl ester” is equivalent to the “alkyl ester or alkenyl ester.” As a result, term such as “R² is an optionally unsaturated alkyl ester (e.g., C(O)—C₁₋₂₀ alkyl, or C(O)—C₁₋₆ alkyl)” are understood to indicate as exemplary esters, both C(O)—C₁₋₂₀ alkyl and C(O)—C₁₋₆ alkyl, as well as C(O)—C₂₋₂₀ alkenyl and C(O)—C₂₋₆ alkenyl. Likewise, the term “R² is mono-unsaturated C(O)—C₇₋₂₀ alkyl” is understood as the same as “R² is mono-unsaturated C(O)—C₇₋₂₀ alkenyl”.

The term “alkenyl” as used herein refers to a monovalent or bivalent, branched or unbranched, unsaturated hydrocarbon group typically although not necessarily containing 2 to about 12 carbon atoms and 1-10 carbon-carbon double bonds, such as ethylene, n-propylene, isopropylene, n-butylene, isobutylene, t-butylene, octylene, and the like.

The term “alkynyl” as used herein refers to a monovalent or bivalent, branched or unbranched, unsaturated hydrocarbon group typically although not necessarily containing 2 to about 12 carbon atoms and 1-8 carbon-carbon triple bonds, such as ethyne, propyne, butyne, pentyne, hexyne, heptyne, octyne, and the like.

The term “aryl” as used herein refers to an aromatic hydrocarbon moiety comprising at least one aromatic ring of 5-6 carbon atoms, including, for example, an aromatic hydrocarbon having two fused rings and 10 carbon atoms (i.e., naphthalene).

By “substituted” as in “substituted alkyl,” “substituted alkenyl,” “substituted alkynyl,” and the like, it is meant that in the alkyl, alkenyl, alkynyl, or other moiety, at least one hydrogen atom bound to a carbon atom is replaced with one or more non-hydrogen substituents, e.g., by a functional group.

The terms “branched” and “linear” (or “unbranched”) when used in reference to, for example, an alkyl moiety of Cₐ to C_{b} carbon atoms, applies to those carbon atoms defining the alkyl moiety. For example, for a C₄ alkyl moiety, a branched embodiment thereof would include an isobutyl, whereas an unbranched embodiment thereof would be an n-butyl. However, an isobutyl would also qualify as a linear C₃ alkyl moiety (a propyl) itself substituted by a C₁ alkyl (a methyl).

Examples of functional groups include, without limitation: halo, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), halocarbonyl (—CO)—X where X is halo), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), mono-substituted C₁-C₂₄ alkylcarbamoyl (—(CO)—NH(C₁-C₂₄ alkyl)), di-substituted alkylcarbamoyl (—(CO)—N(C₁-C₂₄ alkyl)₂), mono-substituted arylcarbamoyl (—(CO)—NH-aryl), thiocarbamoyl (—(CS)—NH₂), carbamido (—NH—(CO)—NH₂), cyano (—C≡N), isocyano (—N⁺≡C⁻), cyanato (—O—C≡N), isocyanato (—O—N⁺≡C⁻), isothiocyanato (—S—C≡N), azido (—N═N⁺═N⁻), formyl (—(CO)—H), thioformyl (—(CS)—H), amino (—NH₂), mono- and di-(C₁-C₂₄ alkyl)-substituted amino, mono- and di-(C₅-C₂₀ aryl)-substituted amino, C₂-C₂₄ alkylamido (—NH—(CO)-alkyl), C₅-C₂₀ arylamido (—NH—(CO)-aryl), imino (—CR═NH where R=hydrogen, C₁-C₂₄ alkyl, C₅-C₂₀ aryl, C₆-C₂₀ alkaryl, C₆-C₂₀ aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), phosphono (—P(O)(OH)₂), phosphonato (—P(O)(O⁻)₂), phosphinato (—P(O)(O⁻)), phospho (—PO₂), phosphino (—PH₂), mono- and di-(C₁-C₂₄ alkyl)-substituted phosphino, mono- and di-(C₅-C₂₀ aryl)-substituted phosphino; and the hydrocarbyl moieties such as C₁-C₂₄ alkyl (including C₁-C₁₈ alkyl, further including C₁-C₁₂ alkyl, and further including C₁-C₆ alkyl), C₂-C₂₄ alkenyl (including C₂-C₁₈ alkenyl, further including C₂-C₁₂ alkenyl, and further including C₂-C₆ alkenyl), C₂-C₂₄ alkynyl (including C₂-C₁₈ alkynyl, further including C₂-C₁₂ alkynyl, and further including C₂-C₆ alkynyl), C₅-C₃₀ aryl (including C₅-C₂₀ aryl, and further including C₅-C₁₂ aryl), and C₆-C₃₀ aralkyl (including C₆-C₂₀ aralkyl, and further including C₆-C₁₂ aralkyl). In addition, the aforementioned functional groups may, if a particular group permits, be further substituted with one or more additional functional groups or with one or more hydrocarbyl moieties such as those specifically enumerated above. For example, the alkyl or alkenyl group may be branched. For example, the “substituent” is an alkyl group, e.g., a methyl group.

As used herein, the term “fragrance composition” means a mixture of fragrance ingredients, e.g., including a Compound 2 or any of 2.1-2.37, or a Compound 3 or any of 3.1-3.38, or Composition 1 or any of 1.1-1.77, or a Composition 2.38-2.51, or a Composition 3.39-3.52, or any mixtures thereof, or a Product Composition (e.g., Product Composition 1, et seq.) or Composition or product 4.1-4.11, any of which may optionally include one or more auxiliary substances if desired, dissolved in a suitable solvent or mixed with a powdery substrate used to provide a desired odor to a product.

The polyether compounds of described herein may be used with e.g., with: perfumes, soaps, insect repellants and insecticides, detergents, household cleaning agents, air fresheners, room sprays, pomanders, candles, cosmetics, toilet waters, pre- and aftershave lotions, talcum powders, hair-care products, body deodorants, anti-perspirants, shampoo, cologne, shower gel, hair spray, and pet litter.

Fragrance and ingredients and mixtures of fragrance ingredients that may be used in combination with the disclosed compound for the manufacture of fragrance compositions include, but are not limited to, natural products including extracts, animal products and essential oils, absolutes, resinoids, resins, and concretes, and synthetic fragrance materials which include, but are not limited to, alcohols, aldehydes, ketones, ethers, acids, esters, acetals, phenols, ethers, lactones, furansketals, nitriles, acids, and hydrocarbons, including both saturated and unsaturated compounds and aliphatic carbocyclic and heterocyclic compounds, and animal products.

As used herein, “citronellol polymer” and “prenol polymer” is meant to include all derivatives and cyclic forms of the citronellol and prenol and polymer.

In the present specification, the structural formula of the compounds represents a certain isomer for convenience in some cases, but the present invention includes all isomers, such as geometrical isomers, optical isomers based on an asymmetrical carbon, stereoisomers, tautomers, and the like. In addition, a crystal polymorphism may be present for the compounds represented by the formula, it is noted that any crystal form, crystal form mixture, or anhydride or hydrate thereof is included in the scope of the present invention.

Where a range is recited, such as 0-10 or 1-7, the range embraces all integer values within the range, as well as integer subranges. Thus, the range 0-10 includes 0-9, 0-8, 0-7, 0-6, 0-5, 0-4, 0-3, 0-2, 1-10, 1-9, 1-8, 1-7, 1-6, 1-5, 1-4, 1-3, 1-2, 1-9, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 4-4, 4-3, 5-10, 5-9, 5-8, 5-7, 5-6, 6-10, 6-9, 6-8, 6-7, 7-10, 7-9, 7-8, 8-10, 8-9, 9-10, 0, 1, 2, 3, 4, 5, 6, 7, 8, 9 and 10.

All percentages used herein, unless otherwise indicated, are by volume.

All ratios used herein, unless otherwise indicated, are by molarity.

### EXAMPLES

### Example 1: Continuous citronellol polymerization over cation exchange resin

A 6-foot-long, 0.25-inch smooth bore stainless steel tube with a 0.01-inch wall thickness is packed with Amberlyst resin and is coiled and outfitted with PTFE tubing on either end for continuous flow. The coil is heated to 50° C. in an oil bath and 300 g of citronellol is pumped through the packed coil at a rate of 2 ml/min. The material coming out of the coil has reached a high degree of polymerization as shown by ¹H-NMR analysis. NMR indicates a dramatic increase in the number of protons associated with methylene groups adjacent to ether oxygen atoms (˜3.3 ppm) compared to the protons associated with methylene groups adjacent to alcohol oxygen atoms (˜3.6 ppm). The integrated ratio is found to be about 1:1 for these two different sets of protons.

Pressurized nitrogen gas is used to expel all material from the coil. The collected material is further distilled under vacuum (0.7 mbar) at elevated temperature (up to 160° C.) to remove monomeric and dimeric species, resulting in a clear, odorless liquid identified as follows:

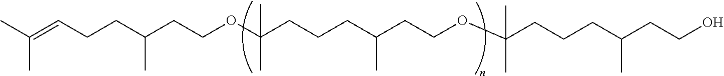

Various fractions may be collected, each having different values for number average molecular weight, weight average molecular weight, polydispersity and values of n. Such fractions have differing physical properties, such as viscosity, refractive index, boiling point, and surface tension.

One fraction is obtained wherein the citronellol polymer has an average value of n of 0-4 (Fraction A). Another fraction is obtained wherein the citronellol polymer has an average value of n of 1-7 (Fraction B).

### Example 2: Citronellol Polymer Acetate

To a citronellol polymer isolated according to the procedure of Example 1 (Fraction A), tetrahydrofuran (THF) and N,N-dimethylaminopyridine (DMAP) are added. The solution is stirred and cooled in an ice bath. Acetic anhydride is added dropwise, and then the solution is allowed to warm to room temperature and is stirred overnight. The reaction is found to be complete by thin layer chromatography (TLC) and it is quenched with the slow addition of water. After stirring for three hours, the THF is removed under reduced pressure on a rotary evaporator and the organic phase is diluted with MTBE and washed with 10% sodium carbonate until the pH is 8. The phases are partitioned, the organic phase is dried with sodium sulfate, and filtered. The material is decolored with activated carbon and further distilled under reduced pressure to remove trace solvent, to yield a clear odorless liquid identified as follows:

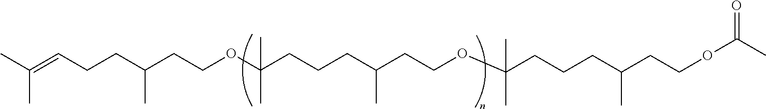

### Example 3: Alternate Process for Acylation of Citronellol Polymer

Acetic anhydride (57 mL) is added to citronellol polymer (102 g), isolated according to the procedure of Example 1, and the reaction temperature is increased slowly to 75° C. The temperature of the reaction is maintained at 75° C. to 80° C. TLC is used to monitor the progress of the reaction. After 3 hours, TLC and NMR show that the reaction is complete. Acetic acid and residual acetic anhydride are removed via distillation to yield 114 g (83%) of citronellol polymer acetate. The product is analyzed and is found to be consistent with the citronellol polymer acetate as obtained according to Example 2.

### Example 4: Determination of Polydispersity of Citronellol Polymer Acetate and Citronellol Polymer

A sample of citronellol polymer acetate of Example 2 is prepared by weighting out 20-25 mg in a 10-mL volumetric flask using the diluent (THF, unstabilized) to make up the volume. The mixture is shaken well until it is homogenous. An isocratic chromatographic condition is used to separate the polymer peaks for analysis. The chromatographic parameters are listed below:

Column: Agilent Oligopore GPC, 6 μm, 3.5 mm×700 mm

Diluent: THF, without BHT inhibitor (unstabilized)

Mobile Phase: THF, without BHT inhibitor (unstabilized)

Flow Rate: 1.0 mL/min

Column Temperature: 25±2° C.

Detection: 220 nm

Injection Volume: 30 μL

Run Time: 25 minutes

Calculation: Because there is the one double bond in each polymer molecule is the only functional group absorbing light at a 220 nm wavelength, the area % values derived from the chromatogram are equivalent to mole % values. Therefore, the HPLC data report values for peak are used for calculation, and only need to be corrected for weight percent calculation.

Mass=(theoretical molecular weight)×(Area %)

Mass %=(mass/sum of mass)×100

Mn (number average)=Area in mAUs/molecular weight

Mw (weight average)=(Area in mAUs)×(molecular weight)

PDI (polydispersity index)=Mw/Mn

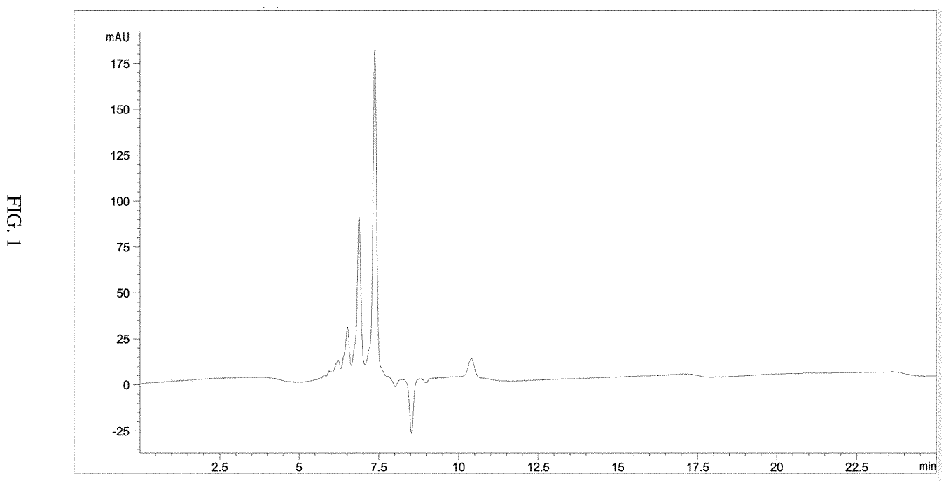

The HPLC chromatogram is shown in FIG. 1. Data values derived from the chromatogram are shown in the table below (n refers to the value of n in polymer formulas described herein):

| Polymer | Retention | Area | Mol. | | Mass |
|---|---|---|---|---|---|
| (n) | Time | (%) | Weight | Mass | % |
| dimer | 7.371 | 60.9931 | 354.58 | 21626.9334 | 49.64 |
| (n = 0) | | | | | |
| trimer | 6.877 | 28.8201 | 510.85 | 14722.74809 | 33.74 |
| (n = 1) | | | | | |
| tetramer | 6.512 | 7.7676 | 667.12 | 5175.1834 | 11.85 |
| (n = 2) | | | | | |
| pentamer | 6.218 | 1.8749 | 823.39 | 1543.773911 | 3.53 |
| (n = 3) | | | | | |
| hexamer | 5.952 | 0.5545 | 979.66 | 543.22147 | 1.24 |
| (n = 4) | | | | | |
| Sum | N/A | 100.0 | N/A | N/A | 100.0 |

The data shows that the primary component of the citronellol polymer acetate composition is the dimer (polymer having the n value 0), present in an estimated amount of 49.64 weight percent. The remaining major components are the trimer, tetramer, hexamer and pentamer, whose total amount is estimated at 50.36 weight percent.

Number average molecular weight, weight average molecular weight, and polydispersity are calculated as shown in the table below:

| | Reten- | | | | |
|---|---|---|---|---|---|
| | tion | Area | Mol. | Area/Mol. | Area × Mol. |
| | Time | (mAU*s) | Weight | Weight | Weight |
| hexamer | 5.952 | 12.60834 | 979.66 | 0.012870118 | 12351.88636 |
| pentamer | 6.218 | 42.63618 | 823.39 | 0.05178127 | 35106.20425 |
| tetramer | 6.512 | 176.40524 | 667.12 | 0.264428049 | 117683.4637 |
| trimer | 6.877 | 655.36853 | 510.85 | 1.28289817 | 334795.0136 |
| dimer | 7.371 | 1386.98315 | 354.58 | 3.911622624 | 491796.4853 |
| Sum | N/A | 2274.00144 | N/A | 5.523600231 | 991733.05321 |

The results provide a calculated Mn for the composition of 411.7, and calculated Mw for the composition of 436.1, and a calculated polydispersity index of 1.06.

The composition has the following physical properties:

Odor: Odorless

Appearance: Thick colorless liquid

Density (g/mL): 0.9134

Viscosity (cP @ 25° C.): 42.97

Refractive index (@ 20° C.): 1.45670

Surface Tension (@ 25° C.): 28.7 mN/m.

Following the same procedure as described above, except using an HPLC method with 18-minute run time, the Citronellol polymer of Example 1 (Fraction B) is submitted to polydispersity determination. The data shows that the primary component of the citronellol polymer Fraction B composition is the trimer (polymer having the n value 1), present in an estimated amount of about 45-57 weight percent. The remaining major components are the tetramer, pentamer, and hexamer, whose total amount is estimated at 42-46 weight percent, with the balance dimer, heptamer and octamer.

The results provide a calculated Mn for the composition of about 521 to 538, and calculated Mw for the composition of about 565 to 581, and a calculated polydispersity index of 1.08 to 1.09.

### Example 5: Hydrating Face Cream

A hydrating face cream is prepared using the citronellol polymer of Example 1, or the citronellol acetate polymer of Example 2 or 3, and a mixture of hydrocarbons, for example, according to the table below (percent values shown are w/w):

| Ingredient | Weight % | Phase |
|---|---|---|
| Water | Q.S. (~81) | A |
| Hyaluronic acid | 0.3 | A |
| Emulsification Blend | 6.0 | B |
| Citronellol polymer (e.g., | 2.25 | B |
| Ex.1, Ex.2 or Ex.3) | | |
| Hydrocarbons (e.g., | 2.25 | B |
| tridecane/undecane mixture) | | |
| Squalene Oil | 3 | B |
| Grapeseed Oil | 3 | B |
| Peony Fragrance Oil | 0.2 | C |
| Vitamin E | 0.5 | C |
| Preservative | 1 | C |

A face cream composition is prepared by mixing hyaluronic acid (Phase A) in water on high sheer in a beaker. The mixture is slowly heated to 50° C. The citronellol polymer (preferably citronellol polymer acetate) is then mixed with the hydrocarbon mixture (preferably Cetiol® emollient, sold by BASF, a mixture of about 20-50 wt. % tridecane and 50-80 wt. % undecane). In a separate beaker measure, the remaining ingredients of Phase B are combined with the citronellol polymer/hydrocarbon mixture, thoroughly mixed, and melted at 75° C. The combined Phase B is added to beaker containing Phase A in water and mixed on high sheer, and then cooled down to ambient temperature. Then Phase C is added and mixed until homogenized. The composition is found to have a pH of 6.03.

It is found that the new composition is a lightweight face cream which leaves the skin feeling hydrated, soft and breathable. It is creamy, plump, soft, hydrating, and supple in nature.

### Example 6: Hair Conditioner

A hair conditioner is prepared by using the citronellol polymer of Example 1, or the citronellol acetate polymer of Example 2 or 3, and a mixture of hydrocarbons, for example, according to the table below (percent values shown are w/w):

| Ingredients | Weight % | Phase |
|---|---|---|
| Water | Q.S. (~86) | A |
| Glycerin | 2.0 | A |
| Behetrimonium | 6.0 | B |
| Methosulfate/Cetearyl | | |
| Alcohol mixture | | |
| Citronellol polymer (e.g., | 2.5 | B |
| Ex.1, Ex.2 or Ex.3) | | |
| Hydrocarbons (e.g., | 2.5 | B |
| tridecane/undecane mixture) | | |
| Gluconolactone, sodium | 1 | C |
| benzoate, calcium gluconate | | |

Phase A is combined and heated to 65-70° C. and mixed thoroughly. The citronellol polymer (preferably citronellol polymer acetate) is then mixed with the hydrocarbon mixture (preferably Cetiol® emollient, sold by BASF, a mixture of about 20-50 wt. % tridecane and 50-80 wt. % undecane). At this temperature the Phase B ingredients, including the citronellol polymer/hydrocarbon mixture, are added one by one until each ingredient is mixed well. It is then homogenized under high shear until fully homogenous. Afterwards, the mixture is cooled under medium speed mixing to about 60° C. At this point, Phase C is added and mixed for about 10 minutes. Finally, it is cooled to 25° C. or at ambient temperature and the pH is adjusted to between 4.5 and 5.5.

It is found that the final composition provides appropriate conditioning and shine.

### Example 7: Hair Milk Spray

A leave-in hair milk spray formulation is prepared by using the citronellol polymer of Example 1, or the citronellol acetate polymer of Example 2 or 3, and a mixture of hydrocarbons, for example, according to the table below (percent values shown are w/w):

| Ingredients | Weight % | Phase |
|---|---|---|
| Water | Q.S. (~89) | A |
| Glycerin | 2.0 | A |
| Behetrimonium | 3.0 | B |
| Methosulfate/Cetearyl | | |
| Alcohol mixture | | |
| Citronellol polymer (e.g., | 2.5 | B |
| Ex.1, Ex.2 or Ex.3) | | |
| Hydrocarbons (e.g., | 2.5 | B |
| tridecane/undecane mixture) | | |
| Gluconolactone, sodium | 1 | C |
| benzoate, calcium gluconate | | |

Phase A is combined and heated up to 65 to 70° C. and mixed thoroughly. The citronellol polymer (preferably citronellol polymer acetate) is then mixed with the hydrocarbon mixture (preferably Cetiol® emollient, sold by BASF, a mixture of about 20-50 wt. % tridecane and 50-80 wt. % undecane). At this temperature the Phase B ingredients, including the citronellol polymer/hydrocarbon mixture, are added one by one until each ingredient is mixed well. It is then homogenized under high shear until fully homogenous. Afterwards, it is cooled under medium speed mixing to at or below 60° C. At this point, Phase C is added and mixed for about 10 minutes. Finally, it is cooled to 25° C. or at ambient temperature and the pH is adjusted to between 4.5 to 5.5.

It is found that this sprayable leave-in conditioning system helps to tame frizz and add some body to flyaway hair that's easy to use throughout the day.

### Example 8: Moisturizing Cream

A moisturizing cream formulation is prepared by using the citronellol polymer of Example 1, or the citronellol acetate polymer of Example 2 or 3, and a mixture of hydrocarbons, for example, according to the table below (percent values shown are w/w):

| Ingredients | Weight % | Phase |
|---|---|---|
| Water | Q.S. (~63) | A |
| Glycerin | 1.0 | A |
| Emulsification Blend | 5.0 | B |
| Citronellol polymer (e.g., | 5.0 | B |
| Ex.1, Ex.2 or Ex.3) | | |
| Hydrocarbons (e.g., | 5.0 | B |
| tridecane/undecane mixture) | | |
| Grapeseed Oil | 10.0 | B |
| Sunflower Oil | 10.0 | B |
| Tocopherol | 0.1 | B |
| Gluconolactone, sodium | 1 | C |
| benzoate, calcium gluconate | | |

Phase A is combined and heated to 65 to 70° C. and mixed. The citronellol polymer (preferably citronellol polymer acetate) is then mixed with the hydrocarbon mixture (preferably Cetiol® emollient, sold by BASF, a mixture of about 20-50 wt. % tridecane and 50-80 wt. % undecane). At this temperature the Phase B ingredients, including the citronellol polymer/hydrocarbon mixture, are added one by one until each ingredient is mixed well. It is then homogenized under high shear. Afterwards, it is cooled under medium speed mixing to at or below 40° C. At this point, Phase C is added and mix for about 10 minutes. Finally, it is cooled to 25° C. or at ambient temperature and the pH is adjusted to between 4.5 and 5.5.

It is found that this moisturizing cream for dry skin leaves skin looking immediately more radiant and feeling unbelievably soft and smooth. It is formulated with oils that are non-comedogenic and quick absorbing to offer amazing nourishment to the skin.

### Example 9: Aerosol Body Spray

An aerosol body spray may be prepared by using the citronellol polymer of Example 1, or the citronellol acetate polymer of Example 2 or 3, a propellant mixture, and a selection of surfactants and other excipients, for example, according to the table below (percent values shown are w/w):

| Ingredients | Weight % |
|---|---|
| Butane | 10-90% |
| Citronellol Polymer | 1-20% |
| 1,1-Difluoroethane | 1-20% |
| Aluminum Chlorohydrate | 0.1-10% |
| Isobutane | 0.1-10% |
| PPG-14 Butyl Ether | 0.1-10% |
| Parfum | 0.01-5% |
| Disteardimonium Hectorite | 0.01-5% |
| Propane | 0.01-5% |
| Butylated Hydroxytoluene | 0.01-5% |
| Propylene Carbonate | 0.01-5% |

### Example 10: Aerosol Sunscreen

An aerosol sunscreen spray may be prepared by using the citronellol polymer of Example 1, or the citronellol acetate polymer of Example 2 or 3, a propellant mixture, and a selection of surfactants and other excipients, for example, according to the table below (percent values shown are w/w):

| Ingredients | Weight % |
|---|---|
| Propellant (e.g., butane, | 40-90% |
| isobutane and/or propane) | |
| Citronellol Polymer | 1-20% |
| UV Absorbers (e.g., | 0.05-10% |
| avobenzone, octisalate) | |
| Photostabilizers (e.g., | 0.05-10% |
| octocrylene) | |
| Denatured alcohol (e.g., | 5-30% |
| SD alcohol) | |
| Polymers (e.g., polyester-27) | 0.1-20% |
| Antioxidants (e.g., | 0.01-5% |
| tocopherol) | |
| Fragrance | 0.01-5% |

### Example 11: Aerosol Hair Spray

An aerosol hair spray may be prepared by using the citronellol polymer of Example 1, or the citronellol acetate polymer of Example 2 or 3, a propellant mixture, and a selection of surfactants and other excipients, for example, according to the table below (percent values shown are w/w):

| Ingredients | Weight % |
|---|---|
| Propellant (e.g., hydrocarbons, | 40-90% |
| hydrofluorocarbons) | |
| Citronellol Polymer | 1-20% |
| Polymers (e.g., polyacrylates, | 0.05-20% |
| polyethylene glycols, polyethylene- | |
| polypropylene copolymers) | |
| Plasticizers (e.g., aminomethyl propanol) | 0.05-20% |
| Surfactants (e.g., panthenyl ethyl ether) | 0.05-20% |
| Denatured alcohol (e.g., SD alcohol) | 0-30% |
| Water | 0-20% |
| Preservatives (e.g., benzyl alcohol, | 0.01-5% |
| benzyl benzoate, benzyl salicylate) | |
| Antioxidants (e.g., tocopherol) | 0.01-5% |
| Fragrances (e.g., linalool, limonene, | 0.01-5% |
| isoeugenol, alpha-isomethyl ionene, | |
| geraniol, butyl phenyl citronellol, butyl | |
| phenyl methylpropional, citral, | |
| amyl cinnamate, coumarin) | |

1. A composition comprising a compound according to Formula I below: 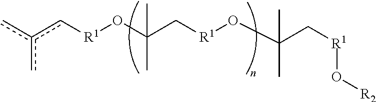
wherein R¹ is optionally substituted C₁-C₁₂ alkyl; R² is H, C₁₋₂₀alkyl, aryl, aryl-C₁₋₂ alkyl, optionally unsaturated alkyl esters or aryl esters, or R² is a moiety: 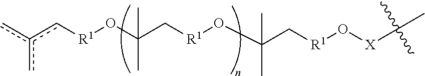
wherein X is a diacyl moiety of formula —C(O)—R³—C(O)—, wherein R³ is optionally
substituted C₁₋₂₂ alkyl, optionally substituted C₂₋₂₂ alkenyl or optionally substituted aryl; and wherein n is an integer between 0 and 10 (e.g., 0 to 4); and wherein the number average or weight average molecular weight of the compounds of Formula I in the composition, exclusive of the group R², is 150 to 2000 Daltons (e.g., 300 to 800 Daltons) and/or wherein the polydispersity (M_{w}/Mₙ) of the compounds of Formula I in the composition (without taking into account the group R²) in the range of 1 to 5.

2. The composition according to claim 1, wherein R¹ is CH₂CH₂CH(CH₃)CH₂CH₂.

3. The composition according to claim 1, wherein R² is H.

4. The composition according to claim 1, wherein R² is C(O)—C₁₋₆ alkyl.

5. The composition according to claim 4, wherein R² is C(O)-methyl.

6. The composition according to claim 1, wherein n is 0, 1, 2, 3, or 4.

7. The composition according to claim 1, wherein the terminal group

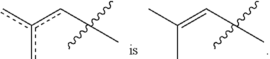

8. The composition according to claim 1, wherein:
the Compound of Formula I is: 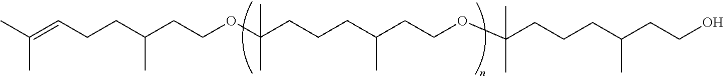
wherein n is 0-10 (e.g., 0, 1 or 2); or
the Compound of Formula I is: 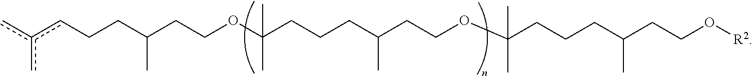
wherein n is 0-10 (e.g., 0, 1 or 2), and wherein R² is —C(O)-methyl.

9. The composition according to claim 8, wherein the composition comprises said Compound of Formula I wherein n is 0 to an extent of greater than 40%, e.g., greater than 50%, greater than 60%, or greater than 70%, or greater than 80%, or greater than 90%, or 40-70% or 40-60%, or 40-50%, said percent being measured either as the number percent of the molecules in the composition or as the weight percent of the total weight of the composition.

10. The composition according to claim 8, wherein the composition comprises one or more Compounds of Formula I wherein each compound independently has a value of n from 0 to 4, and each compound being present in an amount of at least 1% and up to 90%, e.g., 5 to 50%, or 5 to 40% or 5 to 30%, or 5 to 25%, or 5 to 20% or 5 to 15%, or 5 to 10%, or 40 to 50%, or 30 to 40% or 20 to 30%, or 10 to 20%, or 1 to 10%, or 1 to 5%, or 1 to 2%, said percent being measured either as the number percent of the molecules in the composition or as the weight percent of the total weight of the composition.

11. The composition according to claim 8, wherein the compounds of Formula I in the composition have an average n value of about 0, measured either as a weight average or number average, optionally about 0.5.

12. The composition according to claim 1, wherein the number average molecular weight (Mₙ) of the compounds of Formula I in the composition, optionally exclusive of the group R², is 150 to 2000 Daltons; or wherein the weight average molecular weight (M_{w}) of the compounds of Formula I in the composition, optionally exclusive of the group R², is 150 to 2000 Daltons.

13. The composition according to claim 1, wherein the polydispersity (M_{w}/Mₙ) of the compounds of Formula I in the composition (optionally without taking into account the mass of the group R²) is in the range of 1 to 5.

14. The composition according to claim 13, wherein the polydispersity (M_{w}/Mₙ) is in the range of 1 to 2, or 1 to 1.20, or 1 to 1.15, or 1 to 1.10, or about 1.06.

15. A compound of Formula II: 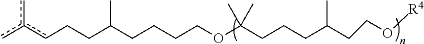
wherein:
R⁴ is selected from Si(C₁₋₄ alkyl)₃, —C(O)—R⁵, —C(O)-A-C(O)—R⁵, -A-C(O)—R⁵, and -A-O—R⁵;
R⁵ is selected from hydrogen, OH, substituted or unsubstituted C₁₋₂₂alkyl, substituted or unsubstituted C₂₋₂₂ alkenyl, substituted or unsubstituted C₂₋₂₂ alkynyl, substituted or unsubstituted C₆₋₁₀ aryl, or wherein R⁵ is a moiety: 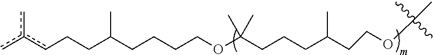
A is selected from substituted or unsubstituted C₁₋₂₀ alkyl, substituted or unsubstituted C₂₋₁₂alkenyl, substituted or unsubstituted C₂₋₁₂ alkynyl, substituted or unsubstituted C₃₋₁₂cycloalkyl, and substituted or unsubstituted C₆₋₁₀ aryl; and
each n and m are independently an integer between 0 and 20;
provided that when R⁴ is —C(O)—R⁵, then R⁵ is not methyl, propyl or oleayl; and
provided that when R⁴ is —C(O)-A-C(O)—R⁵, and R⁵ is a moiety: 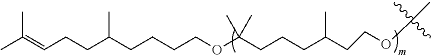
then A is not (CH₂)₄ when n is 0 and m is 1; or
a compound of Formula III: 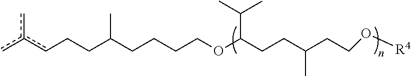
wherein:
R⁴ is selected from hydrogen, Si(C₁₋₄ alkyl)₃, —C(O)—R⁵, —C(O)-A-C(O)—R⁵, -A-C(O)—R⁵, and -A-O—R⁵;
R⁵ is selected from hydrogen, OH, substituted or unsubstituted C₁₋₂₂alkyl, substituted or unsubstituted C₂₋₂₂ alkenyl, substituted or unsubstituted C₂₋₂₂ alkynyl, substituted or unsubstituted C₆₋₁₀ aryl, or wherein R⁵ is a moiety: 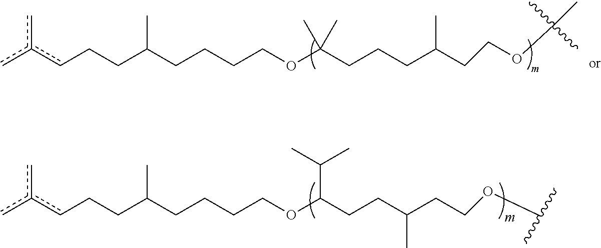
A is selected from substituted or unsubstituted C₁₋₂₀ alkyl, substituted or unsubstituted C₂₋₁₂alkenyl, substituted or unsubstituted C₂₋₁₂ alkynyl, substituted or unsubstituted C₃₋₁₂ cycloalkyl, and substituted or unsubstituted C₆₋₁₀ aryl; and
each n and m are independently an integer between 0 and 20.

16. A product composition which product composition comprises a compound or composition according to claim 1.

17. The product composition according to claim 16, wherein the product is selected from a fragrance product, perfume product, soap product, insect repellant product, insecticide product, detergent product, household cleaning agent product, air freshener product, room spray product, pomander product, candle product, cosmetic product, toilet water product, lotion product, talcum powder product, hair-care product, body deodorant product, anti-perspirant product, shampoo product, pet litter product, topically applied skin care product, paint or coating product, lubricant product, plastic product, defoamer product, hydraulic fluid product, antimicrobial product, crop care product, a product for enhanced oil recovery, fracking and/or other oil field applications, a nail polish remover product, writing ink or printing ink product, an adhesive product, an oral care product, a food product, or a pharmaceutical product.

18. The product composition according to claim 16, wherein the composition further comprises one or more additives selected from: a cooling sensate, a warming sensate and/or a tingling sensate; a flavorant or fragrance; vitamins, minerals, nutraceuticals, energizing agents, soothing agents, coloring agents, amino acids antioxidants, preservatives, pH modifying agents, viscosity adjusting agents, and combinations of any preceding.

19. The product composition according to claim 16 wherein the product composition is an aerosol product.

20. The product composition according to claim 19, wherein the product is an aerosol deodorant, aerosol anti-perspirant, aerosol body spray, aerosol air freshener, aerosol fragrance or perfume product, aerosol insecticide, aerosol lubricant, aerosol hair care product, aerosol sunscreen, aerosol throat analgesic, or aerosol insect repellant.
